(19) United States
(12) Patent Application Publication
(10) Pub. No.: US 2026/0151219 A1
(43) Pub. Date: Jun. 4, 2026

(54) IMPLANTATION AND TRANSPORT SYSTEMS FOR USE WITH MACROENCAPSULATION DEVICES AND RELATED METHODS

(71) Applicant: Vertex Pharmaceuticals Incorporated, Boston, MA (US)

(72) Inventors: Adam MacMillan, Quincy, MA (US); Dharmesh Dilipkumar Patel, Quincy, MA (US); Hans-William Baker, Boston, MA (US); Moses Goddard, Boston, MA (US); James Wilson, Norwood, MA (US); Adam Afzali, Duxbury, MA (US); Neil Bacon, Milton, MA (US)

(73) Assignee: Vertex Pharmaceuticals Incorporated, Boston, MA (US)

(21) Appl. No.: 19/354,991

(22) Filed: Oct. 10, 2025

Related U.S. Application Data
(60) Provisional application No. 63/706,605, filed on Oct. 11, 2024.

(51) Int. Cl. A61F 2/02; A61B 17/34; A61F 2/00; C12M 1/12

(52) U.S. Cl. CPC A61F 2/022; A61B 17/3468; A61F 2/0095; C12M 25/16

(57) ABSTRACT

Macroencapsulation implantation systems and containers for storing and transporting a macroencapsulation device disposed within an end effector as well as related methods are described. In some embodiments, a macroencapsulation implantation system may include a handle that is configured to be selectively engaged with an elongated shaft including an end effector in which a macroencapsulation device may be disposed. In some embodiments, a container may be configured to maintain an end effector, and a macroencapsulation device disposed therein, in a desired pose within an internal volume of the container using an appropriately configured support. In some embodiments, the container may include an appropriately sized and shaped seal between a lid and container such that the container is biased towards an open configuration.

### FIELD

Disclosed embodiments are related to implantation and transport systems for use with macroencapsulation devices and related methods.

### BACKGROUND

Therapeutic devices that deliver biological products can be used to treat metabolic disorders, such as diabetes, in addition to other conditions. These therapeutic devices may be implantable to provide a biological product, such as insulin, for an extended period of time. Some of these devices include macroencapsulation devices which may be used to house cells therein to produce the desired biological product.

### SUMMARY

In one aspect, disclosed herein is a macroencapsulation device storage system comprising a container including an opening and an interior volume, wherein the container includes a lip extending around the opening, and wherein the lip extends out from the opening, a lid configured to be coupled to the opening of the container, wherein the lid includes a groove configured to receive the lip therein when the lid is attached to the container, an elastic seal disposed in the groove of the lid, wherein the lip and the elastic seal are configured to bias the lid towards an open configuration when the elastic seal is compressed, and a support configured to support an end effector containing a macroencapsulation device disposed in the end effector in the container in a predetermined pose.

In one aspect, disclosed herein is a method for storing a macroencapsulation device, the method comprising supporting the macroencapsulation device in a desired pose within an internal volume of a container, inserting a lip extending around an opening of the container into a groove of a lid to compress an elastic seal disposed in the groove of the lid, biasing the lid towards an open configuration when the elastic seal is compressed, and securing the lid to the container.

In one aspect, disclosed herein is a method of handling a macroencapsulation device, the method comprising supporting an end effector including the macroencapsulation device disposed therein in a predetermined pose in an interior volume of a container with a support, applying a first retaining force to the end effector to maintain the end effector in the predetermined pose, connecting the end effector to an implantation system, and applying a force sufficient to overcome the first retaining force to remove the end effector with the macroencapsulation device contained therein from the support while the end effector is connected to the implantation system.

In one aspect, disclosed herein is a support for handling of a macroencapsulation device, the support comprising a base configured to be engaged with a container, an opening extending through the base, a pair of rails positioned on opposing sides of the opening and extending away from the base, wherein the pair of rails include corresponding grooves configured to retain a correspondingly sized and shaped end effector including the macroencapsulation device disposed therebetween, and one or more first retaining tabs configured to selectively engage with the end effector when the end effector is disposed in the support to maintain a predetermined pose of the end effector in the support with a first retaining force.

In one aspect, disclosed herein is an end effector for implanting a macroencapsulation device, the end effector comprising a neck configured to be attached to an elongated shaft of an implantation system, a body attached to and extending out from a distal end portion of the neck, wherein a maximum transverse dimension of the neck in a direction perpendicular to a longitudinal axis of the end effector is less than a maximum transverse dimension of the body in the direction perpendicular to a longitudinal axis of the end effector, a cavity formed in an interior of the body, wherein the cavity is sized and shaped to receive the macroencapsulation device therein, a first opening formed on a proximal end portion of the neck, a channel extending from the first opening to the cavity, wherein the first opening and the channel are configured to receive a pusher of the implantation system inserted therethrough, and an outlet formed on a distal portion of the body, wherein the outlet is sized and shaped to permit the macroencapsulation device to be displaced out of the cavity through the outlet.

In one aspect, disclosed herein is a macroencapsulation device implantation system comprising an elongated shaft including an internal channel extending at least partially through and along a longitudinal axis of the elongated shaft, a handle including a pusher slidably disposed within the internal channel of the elongated shaft, and a first portion of a connection formed on a distal end portion of the elongated shaft, wherein the distal end portion of the elongated shaft and the pusher are configured to be inserted into an opening of an end effector to connect the end effector to the distal end portion of the elongated shaft, and wherein the elongated shaft is configured to be displaced proximally relative to the pusher to displace a macroencapsulation device out of an outlet of a cavity of the end effector with the pusher.

In one aspect, disclosed herein is a method of implanting a macroencapsulation device, the method comprising inserting a distal end portion of an elongated shaft including a first portion of a connection and a pusher into an opening of an end effector including the macroencapsulation device disposed therein, engaging the first portion of the connection with a second portion of the connection formed on the end effector to connect the end effector to the elongated shaft, contacting the pusher with the macroencapsulation device, and proximally displacing the elongated shaft and the end effector relative to the pusher and the macroencapsulation device to displace the macroencapsulation device out of a distal outlet of the end effector.

In one aspect, disclosed herein is a macroencapsulation device implantation system comprising an end effector comprising a neck, a body attached to and extending out from a distal end portion of the neck, wherein a maximum transverse dimension of the neck in a direction perpendicular to a longitudinal axis of the implantation system is less than a maximum transverse dimension of the body in the direction perpendicular to the longitudinal axis of the implantation system, a cavity formed in an interior of the body, wherein the cavity is sized and shaped to receive a macroencapsulation device therein, and an outlet formed on a distal portion of the body, wherein the outlet is sized and shaped to permit the macroencapsulation device to be displaced out of the cavity through the outlet, an elongated shaft, wherein a distal end portion of the elongated shaft is attached to the end effector, and wherein a maximum transverse dimension of the elongated shaft in the direction perpendicular to the longitudinal axis of the implantation system is less than the maximum transverse dimension of the body, an internal channel extending at least partially through and along a longitudinal axis of the elongated shaft to the cavity, and a handle including a pusher slidably disposed within the internal channel of the elongated shaft, wherein the end effector and the elongated shaft are proximally displaceable relative to the pusher.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1D depict one embodiment of an implantation system;

FIGS. 2A-2B illustrate one embodiment of an implantation system when the elongated shaft is in the extended configuration and the retracted configuration respectively;

FIG. 3 illustrates the components of an implantation system in an exploded view according to one exemplary embodiment;

FIGS. 4A-4D depict various views of the top portion of an elongated shaft according to one exemplary embodiment;

FIGS. 5A-5C depict various views of the bottom portion of the elongated shaft;

FIG. 5D depicts the bottom portion of the elongated shaft with the pusher;

FIGS. 6A-6C depict various views of a pusher according to one exemplary embodiment;

FIG. 7A depicts a top perspective view of an end effector according to one exemplary embodiment;

FIG. 7B illustrates an exploded view of the end effector of FIG. 7A;

FIGS. 7C-7F depict various views of the end effector of FIG. 7A;

FIGS. 8A-8C depict various views of a first portion of an end effector according to one exemplary embodiment;

FIGS. 9A-9C depict various views of a second portion of an end effector according to one exemplary embodiment;

FIGS. 10A-10D depict various views of a transport system including a support, end effector, and a macroencapsulation device disposed therein according to one exemplary embodiment;

FIG. 10E depicts a close-up view of the lid, seal, and latching portions of the transport system shown in FIG. 10C;

FIGS. 11A-11D depict various views of an uncovered transport system with a support, end effector, and a macroencapsulation device disposed therein according to one exemplary embodiment.

FIG. 11E depicts a close-up view of an end effector resting on a supporting ledge of a support held in a transport system container as illustrated in FIG. 11C;

FIG. 12 depicts an end effector disposed within a support according to one exemplary embodiment;

FIG. 13A depicts a closed configuration of a wash housing according to one exemplary embodiment;

FIG. 13B depicts an open configuration of a wash housing with end effectors and supports disposed therein according to one exemplary embodiment;

FIG. 14A is a top view of a macroencapsulation device according to one embodiment;

FIG. 14B is a cross-sectional side view of an embodiment of a portion of the membranes of a macroencapsulation device in an unfilled configuration according to one embodiment; and

FIG. 14C is a cross-sectional side view of an embodiment of a portion of the membranes of a macroencapsulation device in a filled configuration according to one embodiment.

### DETAILED DESCRIPTION

Driven by a rising need to deliver biological products to treat various disorders, such as diabetes, different types of implantable therapeutic devices have been engineered. However, such devices, which may include macroencapsulation devices, are often difficult to transport, move, and/or implant within a subject (e.g., a human and/or animal subject). For instance, macroencapsulation devices may include fragile membranes, which may be damaged upon application of sufficiently large forces and/or pressures, and which may cause the overall macroencapsulation device to be damaged and/or contaminated. Due to the therapeutic purposes of the macroencapsulation device, any damage and/or contamination may cause the macroencapsulation device to be unfit for implantation within a subject. Additionally, depending on the type and magnitude of the forces applied to a macroencapsulation device, one or more populations of cells present within the macroencapsulation device may be adversely affected (e.g., through the application of large shear forces which may cause cell injury or death). Further, the Inventors have recognized that many traditional methods and systems used for handling an implantable device during transport and implantation typically do not offer sufficient protection for implantation of macroencapsulation devices that avoid the undesirable application of such forces which may damage the macroencapsulation devices and/or cell populations disposed therein as described above.

In view of the above, the Inventors have recognized the benefits associated with an implantation system for surgical implantation of macroencapsulation devices which may help to minimize forces and/or pressures applied to a macroencapsulation device while transporting, manipulating, and/or implanting a macroencapsulation device. Such transportation systems may also help to minimize handling of the macroencapsulation devices which may also help to reduce contamination of the macroencapsulation devices. Specifically, the Inventors have recognized the benefits associated with storing a macroencapsulation device within a cavity formed in an end effector of a macroencapsulation implantation system prior to implantation. This may include storage and/or transportation of the macroencapsulation device within the end effector. In some embodiments, this end effector including the macroencapsulation device disposed therein may be formed separately from, and may be attachable to, an actuatable portion of the implantation system that may be configured to displace the macroencapsulation device out of the end effector. For example, in some embodiment, an actuatable portion of the implantation system may include an elongated shaft including an internal channel extending at least partially through and along a longitudinal axis of the elongated shaft. In some embodiments, the elongated shaft may be configured to be selectively attached to a proximal portion of the end effector. When attached to the end effector, a pusher slidably disposed within the elongated shaft may include a distal portion of the pusher that extends into the end effector such that the distal portion of the pusher is disposed adjacent to, and in some instances in contact with, the macroencapsulation device. The elongated shaft may be configured to be moved in a proximal direction relative to the pusher and the macroencapsulation device. Accordingly, when the elongated shaft is retracted in the proximal direction, the pusher may maintain a position of the macroencapsulation device as the macroencapsulation device is displaced out of a distal outlet of the end effector from a corresponding cavity of the end effector the macroencapsulation device is disposed in. In other words, in some embodiments, the macroencapsulation device and the pusher may remain stationary during the proximal displacement of the elongated shaft into the handle.

In addition to the above, the Inventors have recognized that prior implantation systems used for implantation of wide macroencapsulation devices exhibited large uniform widths along their length which may cause the devices to be more difficult to reposition within a surgical field during an implantation procedure. Specifically, macroencapsulation devices tend to be wide thin structures. If the implantation system has a correspondingly shaped structure along its entire width, the system may be inserted into a surgical field, but the large width along the entire length may interfere with reorientation of the implantation system within the surgical field. Correspondingly, this may make it difficult to appropriately position a macroencapsulation device during an implantation procedure.

In view of the above, the Inventors have recognized that easy reorientation and repositioning of the macroencapsulation device within a surgical field may be desirable. Specifically, the Inventors have recognized the benefits associated with an elongated shaft of an implantation system including a wider distal portion including the macroencapsulation device disposed therein that is attached to a narrower proximal portion of the elongated shaft. For example, the distal portion of the elongated shaft a macroencapsulation device is disposed in may have a larger transverse dimension than the proximal portion of the elongated shaft.

In some embodiments, the above noted elongated shaft may include an end effector including a cavity that the macroencapsulation device is disposed therein. The end effector may include a distal body including a cavity formed therein and a distal oriented outlet. The cavity may be sized and shaped to receive the macroencapsulation device therein. A neck portion of the end effector may extend proximally from the body, and in some embodiments, may be configured to be attached to a separate portion of the elongated shaft of the implantation system. The body may have a larger transverse dimension than that of the neck. A channel may extend from a proximal opening of the neck through the end effector to the cavity. The channel may be sized and shaped to be aligned with a corresponding channel extending through the elongated shaft and may be configured to accommodate a pusher slidably disposed therein as discussed previously above.

To help limit handling of a macroencapsulation device during transport, storage, and preparation for implantation, it may be desirable to house a macroencapsulation device within a cavity of an end effector of a macroencapsulation implantation system prior to implantation. However, due to size and handling constraints, it may not be desirable to store the macroencapsulation device with the entire associated macroencapsulation implantation system within a desired media. Accordingly, in some embodiments, a macroencapsulation implantation system may include an end effector that is separately formed from and is configured to be selectively attached to an associated elongated shaft of an implantation system. Such an arrangement may permit the end effector and the macroencapsulation device disposed in a cavity therein to be easily stored within an appropriate container with the desired media. The end effector may exhibit the same structure as those embodiments described above and elsewhere in this disclosure. If a handle of an implantation system is intended to be reused, the end effector and elongated shaft of the implantation system may be configured to have a releasable connection such that the elongated handle as well as the associated pusher and handle may be selectively released from an end effector prior to being connected to a separate end effector with another macroencapsulation device disposed therein. Of course, instances in which the connection between the elongated shaft and end effector is configured to be a permanent connection after being engaged are also contemplated.

It should be understood that any appropriate type of connection may be used to selectively connect the end effector and an elongated shaft of an implantation system. Appropriate types of connections may include, but are not limited to, detents, magnetic connections, latches, threaded connections, interlocking mechanical features, and/or any appropriate type of connection as the disclosure is not limited in this fashion. In one embodiment where a releasable configuration is desired, a first portion of a connection, disposed on the elongated shaft, may be a cantilevered hook including a camming surface configured to cam inwards during insertion in an opening of the end effector. The cantilevered hook may be biased into a second portion of the connection formed on the neck of the end effector such as an opening. In instances in which the cantilevered hook is accessible to a user, such as through the opening, the cantilevered hook may be depressed in order to allow for the end effector to be removed from the implantation system. Of course, in embodiments where the end effector is not intended to be removed, the cantilevered hook may not be accessible to a user such that the connection is a permanent connection after it is engaged in the locked configuration.

As mentioned above, a macroencapsulation device may include one or more populations of cells disposed therein. Therefore, during storage and transportation, it may be desirable to store the end effector and macroencapsulation device disposed therein in appropriate media to provide appropriate oxygen and nutrients to the one or more cell populations as well as removal of waste from the one or more cell populations contained within the macroencapsulation device. Accordingly, embodiments including both integrally and separately formed end effectors including a cavity configured to contain a macroencapsulation device disposed therein may include a plurality of slots configured to place the cavity in fluid communication with a surrounding environment (e.g., a liquid media the end effector is disposed in). The plurality of slots may be formed in one or more portions of the end effector configured to be disposed adjacent to a porous portion of the macroencapsulation device when disposed therein. Although the slots may have any appropriate shape and/or orientation, in some embodiments, the slots may be oriented such that a long axis of each slot is oriented parallel to a direction of relative movement between the end effector and the macroencapsulation device during deployment. This may include being parallel to a longitudinal axis of the end effector and/or the overall implantation system. This may help to limit, or avoid, abrasion of the macroencapsulation device over and past edges of the slots during relative movement of the macroencapsulation device and the end effector.

In the various embodiments disclosed herein, the plurality of slots may have a combined area (in a plane parallel to an adjacent surface of the macroencapsulation device) that is greater than or equal to 30%, 40%, 50%, 60%, or other appropriate percentage of a corresponding total surface area of the macroencapsulation device and/or cavity. Accordingly, in some embodiments, the plurality of slots may have a combined area that is greater than or equal to 30% of a corresponding total surface area of the macroencapsulation device and/or cavity. In some embodiments, the plurality of slots may have a combined area that is greater than or equal to 40% of a corresponding total surface area of the macroencapsulation device and/or cavity. In some embodiments, the plurality of slots may have a combined area that is greater than or equal to 50% of a corresponding total surface area of the macroencapsulation device and/or cavity. In some embodiments, the plurality of slots may have a combined area that is greater than or equal to 60% of a corresponding total surface area of the macroencapsulation device and/or cavity. The combined area of the plurality of slots may also be less than or equal to 80%, 70%, 60%, 50%, 40%, or other appropriate percentage of the corresponding total surface area of the macroencapsulation device and/or cavity. Accordingly, in some embodiments, the combined area of the plurality of slots may be less than or equal to 80% of the corresponding total surface area of the macroencapsulation device. In some embodiments, the combined area of the plurality of slots may be less than or equal to 70% of the corresponding total surface area of the macroencapsulation device. In some embodiments, the combined area of the plurality of slots may be less than or equal to 60% of the corresponding total surface area of the macroencapsulation device. In some embodiments, the combined area of the plurality of slots may be less than or equal to 50% of the corresponding total surface area of the macroencapsulation device. In some embodiments, the combined area of the plurality of slots may be less than or equal to 40% of the corresponding total surface area of the macroencapsulation device. Combinations of the above ranges are contemplated including, for example, a combined area of the plurality of slots that is between or equal to 30% and 80% of the corresponding total surface area of the macroencapsulation device and/or cavity.

As noted above, a container may be used to house an end effector with a macroencapsulation device disposed therein within a desired liquid media. However, due to the size of the associated systems, such a container may be large enough such that typical lid attachment constructions may either be bulky, difficult to manipulate, and/or may exhibit large removal forces (e.g., threaded connections with large diameters may have exhibit large opening torques). Additionally, typical seals may also cause the lid to be even more difficult to remove when the container is opened during a surgical procedure. Each of these issues may make it difficult for a user to remove a lid of the system during a surgical procedure. Such operation may be even more difficult when done single handedly which may be desirable in certain surgical applications.

In view of the above, the Inventors have recognized the benefits associated with a combination of a connection between a lid and a container that may be easily unlocked and an elastic seal that biases the lid towards an open configuration when the lid is released. For example, in some embodiments, a container may include an opening fluidly connected to an interior volume of the container. The interior volume may be sized and shaped to receive a support therein in a predetermined pose. Correspondingly, the support may be configured to support an end effector containing a macroencapsulation device therein in a predetermined pose within the support, and thus, within the interior volume of the container. The container may include a lip extending around and out from the opening. The lid includes an elastic seal disposed in a groove of the lid. The elastic seal may be overmolded, separately molded and inserted into the groove, or manufactured in any other appropriate manner. When the lid is coupled to the opening of the container by any appropriate connection, the elastic seal is compressed between the groove of the lid and the lip of the container while the lid is in a closed configuration. In addition to forming the desired liquid seal, when the seal is compressed, the seal may bias the lid towards the open configuration. Thus, when the connection holding the lid and container together in the compressed closed configuration is released, elastic force from the seal may bias the lid towards the open configuration which may help avoid binding of the lid on the container during use. Depending on the desired application, in some embodiments, the lid may be resealable while in other embodiments, the connection used to hold the lid on the container may be single use as the disclosure is not limited in this fashion.

It should be understood that any appropriate type of connection may be used to selectively connect a lid to a container. However, in some embodiments, easily manipulated connections that are configured to compress the lid and seal against the lip of the container may be used. For example, appropriate types of connections that may be used may include, but are not limited to, snaps, clamps, pull tabs, latches, hooks, and/or any other appropriate type of connection. In one specific embodiment, and as elaborated on in the figures, the connection may include a plurality of tabs connected to the lid by corresponding living joints. The tabs may include hooks, barbs, or other appropriate structure that are configured to form a connection with corresponding mechanical features, such as a ledge, formed on the container.

To facilitate the formation of a large sealing force while also biasing the lid towards an open configuration, in some embodiments, the elastic seal may form a wedge seal with the lip of the lid. Specifically, in some embodiments, an interface between the elastic seal and the lip of the container when engaged may be oriented at a desired angle to increase the sealing force while still permitting some of the force to be directed in a direction that biases the lid to the open configuration. For example, the interface between the elastic seal and the lip of the container when engaged may be angled relative to an axis passing through and perpendicular to a plane of the opening. In some instances, this may correspond to a vertical direction relative to a local direction of gravity when a base of the container is disposed on a level supporting surface. While any appropriate angle may be used, in some embodiments, the interface may be oriented at an angle relative to the axis and/or vertical direction that is between or equal to 5° and 45°. In some embodiments, the angle may be between or equal to 5° and 40°. In some embodiments, the angle may be between or equal to 10° and 35°. In some embodiments, the angle may be between or equal to 15° and 30°. In some embodiments, the angle may be between or equal to 20° and 25°. Depending on the embodiment, the above noted interface with the above noted angular ranges may be oriented inwards such that a line normal to and extending away from the lip at the interface is oriented at least partially towards the opening. Of course, embodiments in which the interface is oriented outwards in the opposite direction away from the opening are also contemplated.

As discussed above, macroencapsulation devices are fragile and susceptible to damage during transportation and handling. Thus, the Inventors have recognized that it may be desirable to minimize the directly handling and manipulation of a macroencapsulation device during transport, storage, and preoperative handling. Similarly, the Inventors have recognized that it is desirable to avoid unintentional movement of an end effector within a container during storage and transport to avoid potential damage to the macroencapsulation device as well as a need to rearrange the end effector and/or macroencapsulation device prior to implantation.

In view of the above, it may be desirable for a container to include a support configured to maintain an end effector in a predetermined pose within an interior volume of a container. For example, a support may include a base configured to be engaged with a container to hold the support and the associated end effector in a desired pose within the interior volume of the container. The support may include a pair of rails including groves that are sized and shaped to engage with and retain opposing sides of the end effector to retain the end effector in the support. To facilitate insertion of the end effector into the rails, the rails may be positioned on opposing sides of an opening formed in and extending through the base. While the rails and opening of the base may position the end effector approximately in a desired configuration, end effectors including narrower neck portions may still be able to move within the support. Accordingly, in some embodiments, one or more retaining tabs formed on the support may be configured to selectively engage with the end effector when disposed in the support to maintain a predetermined pose of the end effector in the support with a first retaining force. This first retaining force may be less than a connection force between the end effector and a corresponding elongated shaft of an implantation system when connected.

In some embodiments, a support used to hold an end effector in a desired pose within a container may also be configured for use with a wash container used to apply one or more liquid treatments to a macroencapsulation device prior to implantation. In some such embodiments, a support housing an end effector with a macroencapsulation device disposed therein, may be configured to be removed from a container and inserted in a wash container where one or more liquid washes such as sterile saline, buffer, or other cell media may be used to wash the one or more macroencapsulation devices. For example, a container may include one or more receptacles configured to receive one or more corresponding supports therein. Of course, it may be desirable to facilitate attachment of the end effector to an elongated shaft of an implantation system and removal from the support. Accordingly, in some embodiments, the one or more supports may be configured to be connected to the wash container by one or more corresponding connections. These connections may have a second retaining force that is greater than the first retaining force used to maintain each end effector in the corresponding support and is less than a connection force between an end effector and attached elongated shaft or other portion of an implantation system. Thus, the end effector may be removed from the support with an implantation system while the support is retained in the wash container. Any appropriate type of connection may be used to connect a support to a receptacle of a wash container. For example, appropriate connections may include, but are not limited to, latches, snap connections, magnetic connections, threaded fasteners, mechanical interfering features, and/or any other appropriate type of connection exhibiting the desired combination of retention forces. between the wash container, end effector, and support.

The use of an end effector that is selectively attachable to an elongated shaft of an implantation system for both delivering and storing a macroencapsulation device may offer multiple benefits. For example, reduced handling of the macroencapsulation device may help minimize a potential for contamination and/or damage of the macroencapsulation device both prior to and during implantation. For example, storage, transportation, and delivery from within a cavity of the end effector may obviate the need for a medical practitioner to directly manipulate and/or load the macroencapsulation device prior to implantation while also helping to shield the macroencapsulation device from the inadvertent application of potentially damaging forces and/or contacts. Of course, it should be understood that the current disclosure is not limited to just these benefits and other benefits different from those noted above are also possible. The disclosed implantation systems may also offer improved positioning and handling of a macroencapsulation device during an implantation procedure. Additionally, while a majority of the disclosed embodiments are directed to systems including separately formed end effectors and elongated shafts, instances in which an integrally formed end effector forms a distal portion of the elongated shaft of an implantation system are also contemplated. Of course, it should be understood that the disclosed methods and system may exhibit other benefits different from those noted above as the disclosure is not limited in this fashion.

Appropriate materials for use with any one of the embodiments of a macroencapsulation implantation system and/or associated container disclosed herein may include but are not limited to a biocompatible plastics, metals, ceramics, and/or combinations of the forgoing capable of being used for the applications disclosed herein. For example, suitable materials may include, but are not limited to, aluminum, titanium, stainless steel, alumina, silicone, polycarbonate, polyvinylchloride (PVC), polypropylene (PP), polyether ether ketone (PEEK), polyurethane (PU), and polyethylene (PE). In some embodiments, components of the macroencapsulation implantation system and associated container may also be treated and/or coated to modify properties of the material, such as chemical resistance and/or color. For example, components of the macroencapsulation implantation system and/or associated container constructed at least in part from aluminum may include an anodized coating. It should be understood that the various components of macroencapsulation implantation system and the associated containers may be made from any appropriate combination of materials as the disclosure is not limited to being made from any specific material.

In some embodiments, a macroencapsulation implantation system and/or an associated container is sterile. The macroencapsulation implantation system and/or associated container may be configured as a single-use sterile device or as a reusable sterilizable device. A reusable device may be used multiple times on the same subject and/or it may be sterilized in between each use, especially in between different uses for different subjects. In some embodiments, only a portion of the macroencapsulation implantation system, e.g., the handle, may be reusable and sterilizable, while other components, e.g., the end effector, may be single use. Additionally, some components of the macroencapsulation implantation system, such as the handle, may not directly contact a biological material in some embodiments, and may be removably attachable to a pusher and/or elongated shaft, which may directly contact a biological material. The Inventors have recognized that such a releasable coupling between these components may permit the use of separate sterile components with a single handle in some applications.

For any reusable component which is sterilizable, the component may be made from any appropriate material, including those noted above, for a desired type of sterilization. Possible sterilization methods may include but are not limited to heat sterilization, chemical sterilization, and/or radiation sterilization which include methods such as moist heat (autoclave), dry heat, flash steam, performic acid, peracetic acid, formaldehyde, carbon dioxide, ethylene oxide, ozone, plasma, and ultraviolet light.

A macroencapsulation device may include multiple layers of membranes. At least one exterior membrane of these multiple layers of membranes may be semipermeable. However, embodiments in which each of the membranes is semipermeable or where at least one of the membranes within a device are substantially impermeable are also contemplated. Further, a device may include two stacked membranes, three stacked membranes, and/or any other appropriate number of membranes as the disclosure is not limited in this fashion. For example, in one embodiment including two membranes, either membrane may be semipermeable and the other impermeable or both may be semipermeable. Accordingly, it should be understood that the current disclosure is not limited to any particular combination of membranes within a stacked structure. Exemplary macroencapsulation devices include, for example, those described in WO2018232180, WO2019068059, WO2020206150, WO2020206157, and WO2023023006, each of which is incorporated-by-reference in its entirety.

In some embodiments, a macroencapsulation device may include at least one population of cells disposed within an internal volume of the device. For example, the population of cells may be disposed within an internal volume formed between two or more opposing layers of one or more exterior membranes of the device where an exterior edge of the internal volume may be defined by one or more bonds extending around at least a portion, and in some instances an entire, perimeter of the membranes or other appropriate portion of the membranes. In such an embodiment, at least the exterior membranes of the device may be configured to block passage of the one or more populations of cells out of the device. Accordingly, the one or more populations of cells may be retained within the interior volume of the device. While the use of two exterior membranes forming a single internal volume is primarily described, the use of multiple intermediate membranes positioned between the exterior membranes of a device and/or multiple unconnected interior volumes within a device are also contemplated. Additionally, instances in which a single membrane is folded over and bonded to itself to provide two opposing membranes to form the interior volume are also contemplated.

Although expanded polytetrafluoroethylene (ePTFE) may be used as a membrane material, the membranes of a macroencapsulation device may be formed from any appropriate porous biocompatible material. For example, the various membranes and/or membrane layers, may be formed from any appropriate porous polymeric membrane material. The biocompatible material may be substantially inert towards cells housed within the macroencapsulation device and the surrounding tissue. The biocompatible material may comprise a synthetic polymer or a naturally occurring polymer. In some embodiments, the polymer may also be a linear polymer, a cross linked polymer, a network polymer, an addition polymer, a condensation polymer, an elastomer, a fibrous polymer, a thermoplastic polymer, a non-degradable polymer, combinations of the foregoing, and/or any other appropriate type of polymer as the disclosure is not limited in this fashion. As noted above, in one embodiment, a polymer may comprise expanded polytetrafluoroethylene (ePTFE). Appropriate types of polymers may also comprise polyvinylchloride (PVC), polyethylene (PE), polypropylene (PP), polymethylmethacrylate (PMMA), polystyrene (PS), polytetrafluoroethylene (PTFE), polyurethane (PU), polyamide (nylon), polyethyleneterephthalate (PET), polyethersulfone (PES), polyetherimide (PEI), polyvinylidene difluoride (PVDF), polycaprolactone (PCL), poly(lactic-co-glycolic acid) (PLGA), poly-L-lactide (PLLA), polyacrylonitrile (PAN), electrospun PAN/PVC, any combination of the foregoing, and/or any other appropriate polymeric material. In some embodiments, a membrane used with any of the embodiments disclosed herein may comprise PVDF. In some embodiments, a membrane used with any of the embodiments disclosed herein may comprise electrospun PAN PVC. In some embodiments, a membrane used with any of the embodiments disclosed herein may comprise PES. In some embodiments, a membrane used with any of the embodiments disclosed herein may comprise PS. In some embodiments, a membrane used with any of the embodiments disclosed herein may comprise PAN. In some embodiments, a membrane used with any of the embodiments disclosed herein may comprise polycarbonate. In some embodiments, a membrane used with any of the embodiments disclosed herein may comprise polypropylene. In some embodiments, a membrane used with any one of the embodiments disclosed herein may comprise PVC. In some embodiments, a membrane used with any one of the embodiments disclosed herein may comprise PU. In some embodiments, a membrane used with any one of the embodiments disclosed herein may comprise PET. In some embodiments, a membrane used with any one of the embodiments disclosed herein may comprise PCL. In some embodiments, a membrane used with any one of the embodiments disclosed herein may comprise PLGA. In some embodiments, a membrane used with any one of the embodiments disclosed herein may comprise PLLA. In some embodiments, a membrane used with any one of the embodiments disclosed herein may comprise PMMA. In some embodiments, a membrane used with any one of the embodiments disclosed herein may comprise PEI. In some embodiments, a membrane used with any one of the embodiments disclosed herein may comprise nylon. In some embodiments, a membrane used with any one of the embodiments disclosed herein may comprise PTFE. In some embodiments, a membrane used with any one of the embodiments disclosed herein may comprise ePTFE. In some embodiments, a membrane used with any one of the embodiments disclosed herein may comprise PE. The synthesis methods used for forming one or more of the porous membranes from the above noted polymeric materials may include, but are not limited to, expansion, solvent-casting, immersion precipitation and phase separation, electrospinning, methods that yield isoreticular networks, methods that yield trabecular networks, or any other appropriate method of forming a porous polymer membrane.

Sintering of a membrane may be used to alter the porosity and flux properties of a membrane. For example, the sintering may increase the porosity of the membrane while maintaining its pore structure. The sintering may also improve the mechanical stability and diffusive flux of the membrane. In some instances, a sintered membrane can have a lower melting temperature than an unsintered membrane of the same type. Further, sintered membranes may exhibit a different energy release during a differential scanning calorimetry scan, indicating a more relaxed structure in addition to the thickened porous network exhibited in sintered materials.

In view of the above, sintering may be used to alter the porosity and/or mechanical properties of the membranes, which in turn can be used to tune the porosity and the flux properties of the macroencapsulation device. Accordingly, in some embodiments, any desired combination of sintered and/or unsintered membranes or membrane layers may be used. For instance, two exterior membrane layers of a device may be bonded together where either a sintered and unsintered membrane are bonded together, two sintered membranes are bonded together, or two unsintered membranes are bonded together. Further, any number of intermediate membranes positioned between these exterior membranes may be used where these intermediate membranes may be sintered or unsintered.

The membranes of a macroencapsulation device as described herein may be made from porous membrane materials that are configured to allow for transport through the membranes of materials, such as a biological product, with a molecular weight less than about 3000 kDa, 2000 kDa, 1000 kDa, 500 kDa, 400 kDa, 300 kDa, 200 kDa, 100 kDa, 50 kDa, 40 kDa, 30 kDa, 20 kDa, 10 kDa, 6 kDa, 5 kDa, 4 kDa, 3 kDa, 2 kDa, 1 kDa, and/or any other appropriate range of molecular weights depending on the desired application. The membranes of a macroencapsulation device as described herein may be made from porous membrane materials that are configured to allow for transport through the membranes of materials, such as a biological product, within the molecular weight range of 1-3000 kDa, 1-2000 kDa, 1-1000 kDa, 1-500 kDa, 1-400 kDa, 1-300 kDa, 1-200 kDa, 1-100 kDa, 1-50 kDa, 1-40 kDa, 1-30 kDa, 1-20 kDa, 1-10 kDa, 1-6 kDa, 1-5 kDa, 1-4 kDa, 1-3 kDa, or 1-2 kDa. For example, the one or more membranes of a macroencapsulation device may be configured to permit the flow of insulin through the membranes which has a molecular weight of about 5.8 kDa. In some embodiments, the one or more membranes of a macroencapsulation device may be configured to permit the flow of materials, such as a biological product, within the range of 1-10 kDa. In some embodiments, the one or more membranes of a macroencapsulation device may be configured to permit the flow of materials, such as a biological product, within the range of 1-6 kDa. In some embodiments, the one or more membranes of a macroencapsulation device may be configured to permit the flow of materials, such as a biological product, within the range of 1-5 kDa. In some embodiments, the one or more membranes of a macroencapsulation device may be configured to permit the flow of materials, such as a biological product, within the range of 1-4 kDa. In some embodiments, the one or more membranes of a macroencapsulation device may be configured to permit the flow of materials, such as a biological product, within the range of 1-3 kDa. In some embodiments, the one or more membranes of a macroencapsulation device may be configured to permit the flow of materials, such as a biological product, within the range of 1-2 kDa.

To provide the desired selectivity, the porous membranes used with the macroencapsulation devices disclosed herein may have an open porous structure (i.e., a structure including a plurality of interconnected pores) with average pore sizes that are greater than or equal to about 1 nm, 5 nm, 10 nm, 15 nm, 20 nm, 30 nm, 40 nm, 50 nm, 60nm, 70 nm, 80 nm, 90 nm, 100 nm, 200 nm, 300 nm, and/or any other appropriate size range. Correspondingly, the average pore size of the various membranes described herein may have an average pore size that is less than or equal to 2500 nm, 2000 nm, 1700 nm, 1500 nm, 1400 nm, 1300 nm, 1200 nm, 1100 nm, 1000 nm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, and/or any other appropriate size range. Combinations of the foregoing are contemplated including, for example, an average pore size that is between or equal to 1 nm and 20 nm, 1 nm and 2500 nm, 50 nm and 1200 nm, and/or any other appropriate combination. In some embodiments, the average pore size of the various membranes described herein is between 25 nm and 1500 nm. In some embodiments, the average pore size of the various membranes described herein is between 50 nm and 1200 nm. In some embodiments, the average pore size of the various membranes described herein is between 50 nm and 1000 nm. In some embodiments, the average pore size has an upper size limit of 1500 nm. In some embodiments, the average pore size has an upper size limit of 1200 nm. In some embodiments, the average pore size has a lower size limit of 25 nm. In some embodiments, the average pore size has a lower size limit of about 50 nm. While specific average pore sizes are described above, it should be understood that any appropriate average pore size may be used for the various membranes described herein including average pore sizes both greater than and less than those noted above.

In some embodiments, a cell population contained within a compartment of a macroencapsulation device may be an insulin secreting cell population. In some embodiments, a cell population contained within a compartment of a macroencapsulation device comprises a heterogeneous population of cells. In some embodiments, the cell population comprises at least one cell derived from a stem cell derived cell. In some embodiments, at least one cell, and in some embodiments a majority of the cells are, genetically modified cells. In some cases, at least one cell is genetically engineered to reduce an immune response in a subject upon implantation of the device, as compared to comparable cells that are not genetically engineered. In some embodiments, the cell population is a stem cell derived cell that is capable of glucose-stimulated insulin secretion (GSIS). For example, an appropriate population of cells may comprise pancreatic progenitor cells, endocrine cells, beta cells, a matrix including one or more of the foregoing, or any combination thereof. Further, a matrix may comprise isolated islet cells, isolated cells from pancreas, isolated cells from a tissue, stem cells, stem cell-derived cells (e.g., stem cell-derived islet cells), induced pluripotent cells, differentiated cells, transformed cells, or expression systems, which can synthesize one or more biological products. In some embodiments, the macroencapsulation device comprises a population of stem cell-derived islet cells. In some embodiments, the stem cell-derived islet cells comprise stem cell-derived beta cells, stem cell-derived alpha cells, and/or stem cell-derived delta cells.

To provide sufficient strength and/or rigidity for a macroencapsulation device, the various membranes and frames may be made from materials that are sufficiently stiff to maintain a desired shape of the macroencapsulation device during use. The desired stiffness may be provided via an appropriate combination of a material's Young's modulus (also referred to as an Elastic modulus), thickness, and overall construction which may be balanced with a desired permeability of the device. Appropriate Young's moduli for the various membranes and frames described herein may be at least 10⁵ Pa, 10⁶ Pa, 10⁷ Pa, 10⁸ Pa, 10⁹ Pa, and/or 10¹⁰ Pa. Other appropriate Young's moduli for the various membranes and frames described herein may be used including moduli both greater than and less than these ranges. Ranges between the foregoing Young's moduli are contemplated including, for example, a Young's modulus between or equal to about 10⁶ Pa and 10¹⁰ Pa.

The frame of a macroencapsulation device may be formed from any appropriate biocompatible thermoplastic material. As previously noted, in some embodiments, an appropriate material for the frame may include polyetheretherketone (PEEK). Appropriate materials for the frame may also include, but are not limited to polycarbonate, polyurethane, polyetheretherketone (PEEK), Polyvinyl Chloride (PVC), poly(oxymethylene), poly(methyl methacrylate) (PMMA), thermoplastic polymer based composites, polypropylene, fluorinated ethylene propylene (FEP), low density polyethylene (LDPE), high density polyethylene (HDPE), ultra-high density polyethylene (UHDPE), polycaprolactone, poly(lactide), poly(glycolic acid), poly lactide-co-glycolide, ethylene vinyl acetate copolymer, polyamides, poly(butylene)therephthalate, combinations of the foregoing, and/or any other appropriate thermoplastic material. In addition to the use of a thermoplastic material in a frame, embodiments in which a frame includes a thermoplastic portion configured to be bonded to a membrane and another non-thermoplastic portion are also contemplated as the disclosure is not limited to frames made completely from a thermoplastic material. In some embodiments, an appropriate material for the frame includes polypropylene. In some embodiments, an appropriate material for the frame includes fluorinated ethylene propylene (FEP). In some embodiments, an appropriate material for the frame includes ultra-high density polyethylene (UHDPE). In some embodiments, an appropriate material for the frame includes polycarbonate. In some embodiments, an appropriate material for the frame includes polyurethane. In some embodiments, an appropriate material for the frame includes PVC. In some embodiments, an appropriate material for the frame includes poly(oxymethylene). In some embodiments, an appropriate material for the frame includes poly(methyl methacrylate (PMMA). In some embodiments, an appropriate material for the frame includes thermoplastic polymer based composites. In some embodiments, an appropriate material for the frame includes polypropylene. In some embodiments, an appropriate material for the frame includes LDPE. In some embodiments, an appropriate material for the frame includes HDPE. In some embodiments, an appropriate material for the frame includes polycaprolactone. In some embodiments, an appropriate material for the frame includes poly (lactide). In some embodiments, an appropriate material for the frame includes poly(glycolic acid). In some embodiments, an appropriate material for the frame includes poly lactide-co-glycolide. In some embodiments, an appropriate material for the frame includes ethylene vinyl acetate copolymer. In some embodiments, an appropriate material for the frame includes polyamides. In some embodiments, an appropriate material for the frame includes poly(butylene)therephthalate. In other embodiments, an appropriate material for the frame or portion of the frame may include titanium, graphene, stainless steel, or other appropriate biocompatible material exhibiting sufficient rigidity to function as a frame for the macroencapsulation device.

As used herein, a user of a macroencapsulation implantation system may refer to an individual who may store, transfer, implant, and/or otherwise manipulate the macroencapsulation device either prior to or during a surgical procedure. In some embodiments, a user may refer to a surgeon and/or medical practitioner, and the macroencapsulation implantation system may be used during surgery and/or other medical procedures.

As used herein, a pose may refer to a combination of a position (i.e., three-dimensional position) and orientation (i.e., an angular orientation) of an object. For example, a pose of an elongated shaft disposed within an internal volume of a container may correspond to both the position and orientation of an elongated shaft within an internal volume of a container.

As used herein, proximal and distal designate different portions and directions of operation for an impanation device. For example, the handle of an implantation system may be located on a proximal end portion of the implantation system while the end effector is located distally from the handle of implantation system.

As used herein, the longitudinal axis of an implantation system is the axis extending between the proximal and distal end portions of the implantation system. Accordingly, in some embodiments, an elongated shaft may be configured to be displaced relative to the pusher in a direction that is parallel to the longitudinal axis. As used herein, a transverse dimension or direction of the system is perpendicular to the longitudinal direction and longitudinal axis. In some embodiments, a maximum transverse dimension of a component may be referred to as a width of the component and a minimum transverse dimension of the component may be referred to as a thickness of the component.

It should be understood that the above noted materials, parameter ranges, and general description of the construction and/or operation of various components may be used either individually or in combination with one another with any one of the embodiments of a macroencapsulation implantation system and/or container disclosed herein.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

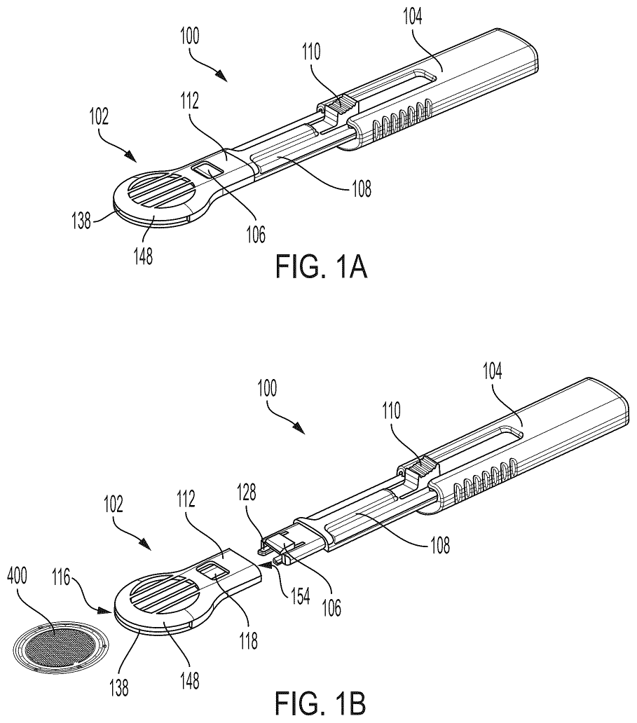

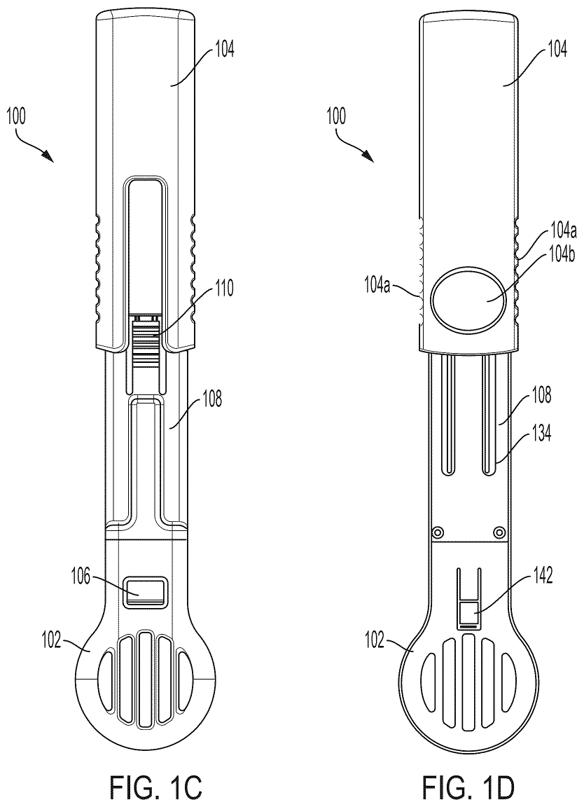

FIGS. 1A-1D depict the overall structure of one embodiment of an implantation system 100. FIG. 1A depicts a prospective view of the entire implantation system. Starting on the proximal side, the embodiment depicted in FIG. 1A includes a handle 104 configured to be grasped by a user's hand. For example, as shown in FIGS. 1C and 1D, the handle may include one or more handle grips 104a and 104b formed therein that may be configured to assist a user in grabbing and handling the handle 104. An elongated shaft 108 extends distally from the handle 104. An end effector 102 is disposed on and is connected to a distal end portion of the elongated shaft 108. While the end effector 102 is depicted as a separately formed structure that is selectively connected to the elongated shaft 108, embodiments in which the end effector 102 is integrally formed with and/or is part of the elongated shaft 108 are also contemplated.

As seen in FIG. 1B and the other figures described herein, the implantation system 100 includes a pusher 128 that extends distally from the handle 104 through a channel formed in the elongated shaft 108 such that the pusher 128 extends out from a distal end of the elongated shaft 108 and into the end effector 102. As elaborated on further below, and as illustrated in FIG. 1B, a macroencapsulation device including one or more cell populations disposed therein, may be received in a cavity 116 of the end effector 100. The end effector 102 includes a distal outlet 138 that is connected with the cavity 116 such that the pusher 128 may be used to displace the macroencapsulation device 400 out of the end effector 102 through the outlet 138 when the elongated shaft 108 and end effector 102 are displaced relative to the pusher 128 and handle 104 another. Thus, the outlet 138 may be appropriately sized and shaped to permit the macroencapsulation device 400 to be displaced out of the cavity 116 through the outlet 138.

As mentioned above, an implantation system with a uniform transverse dimension may be difficult to reposition which may hinder the implantation of a macroencapsulation device 400 in a desired location. Accordingly, it may be desirable for the distal portion or body 148 of the end effector 102 including the macroencapsulation device 400 disposed therein to be wider than a portion of the elongated shaft 108 connected to a neck 112 of the end effector 102. In other words, a maximum transverse dimension of the body 148 of the end effector 102 may be greater than a maximum transverse dimension of the neck 112 extending proximally from the body 148 as well as the portion of the elongated shaft 108 connected to the neck 112. In some embodiments, a width of the elongated shaft 108 may be less than a width of the body 148 along an entire length of the elongated shaft 108. In some embodiments, a ratio of a maximum transverse dimension of the neck 112 adjacent to the elongated shaft and/or a portion of the elongated shaft 108 attached to the end effector 102 with a maximum transverse dimension of the body 148 of the end effector 102 may be between or equal to about 0.25 and 0.75. When combined with the rounded or semicircular shape of the body 148, this overall shape and sizing may permit easy repositioning of the macroencapsulation device within a surgical field by a user.

It should be understood that the disclosed implantation systems 100 and macroencapsulation devices 400 may exhibit any desired combination of dimensions depending on the specific application. However, in some embodiments, portions of an implantation system configured to be inserted into a subject (e.g., an end effector 102 and/or elongated shaft 108) may have a thickness (i.e., a minimum transverse dimension) that is between or equal to about 0.5 cm and 1.0 cm. The portions of the implantation system 100 configured to be inserted into a subject may also have a width (i.e., a maximum transverse dimension) that is between or equal to about 1 cm and 5 cm. In some embodiment, the width may be between or equal to about 3 cm and 5 cm. Additionally, the portions of the implantation system 100 configured to be inserted into the subject may have a combined length extending along a longitudinal axis of the system (e.g., a combined length of an end effector 102 and portion of an elongated shaft 108 extending from a handle 104) that is between or equal to about 10 cm and 30 cm. In some embodiments, a ratio of a width (i.e., a maximum transverse dimension) of the body 148 of an end effector 102 to a thickness of the body 148 may be equal between or equal to about 5 and 20. In some embodiments, the thickness of the body 148 may be substantially similar to the thickness of the macroencapsulation device 400.

FIG. 1B depicts the implantation system 100 with the end effector 102 and macroencapsulation device 400 in an exploded view with the end effector 102 separated from the elongated shaft 108. In the depicted embodiment, the implantation system includes a connection that is used to selectively connect the end effector 102 to the distal portion of the elongated shaft 108. Specifically, the elongated shaft 108 includes a first portion of a connection 106 associated with the distal portion of the elongated shaft 108. The first portion of the connection 106 is configured to be connected to a second portion of the connection 118 associated with the neck 112 of the end effector 102 when a distal portion of the elongated shaft 108 is inserted into a correspondingly sized and shaped opening 154 formed on a proximal face of the end effector 102 oriented towards the elongated shaft 108 when the end effector 102 and elongated shaft are connected. The connection may be configured to maintain the end effector 102 and elongated shaft 108 in a desired connected configuration during use.

In the depicted embodiment, the first portion of the connection 106 is a cantilevered catch including one or more camming surfaces disposed on a distal end portion of a distally extending tab of the elongated shaft that when inserted into the proximally oriented opening 154 of the end effector 102 are cammed inwards to permit insertion of the elongated shaft 108. When fully inserted, the cantilevered catch may cam outwards into an opening formed in the neck 112 of the end effector 102. In some embodiments, the opening may extend through a side wall of the neck 112 such that the first portion of the connection 106 (i.e., the cantilevered latch) is accessible to a user such that it may be depressed so an operator may manually unlock and remove the end effector 102. However, embodiments in which the second connection 118 is a correspondingly sized and shaped feature that does not extend through the neck 112 to an exterior surface to form a permanent connection are also contemplated.

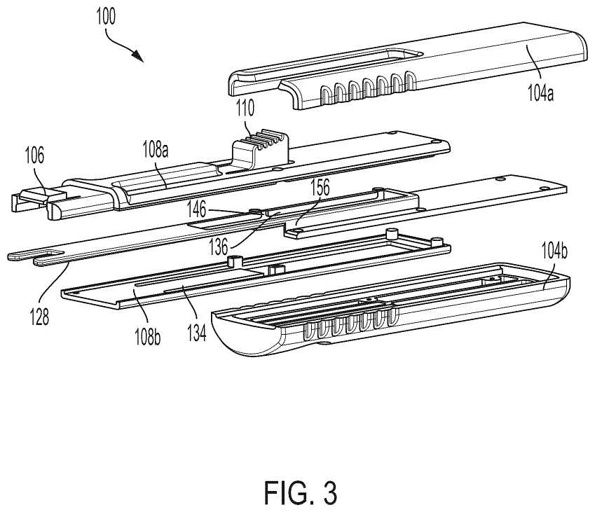

It should be understood that while a connectable end effector 102 and elongated shaft 108 are described above and shown in the figures, the disclosure is not so limited. For example, in some embodiments, an implantation system 100 may include an end effector 102 that is integrally formed with and/or is permanently attached to the elongated shaft 108. Such a system may operate in similar manner to the methods of operation described for an implantation system 100 including a removable end effector 102. Lastly, FIG. 1D also depicts guides 134 which are slots formed in the elongated shaft 108. The guides may assist in guiding the elongated shaft to slide proximally along a desired direction and within a desired range of movement as elaborated on further below relative to FIG. 3.

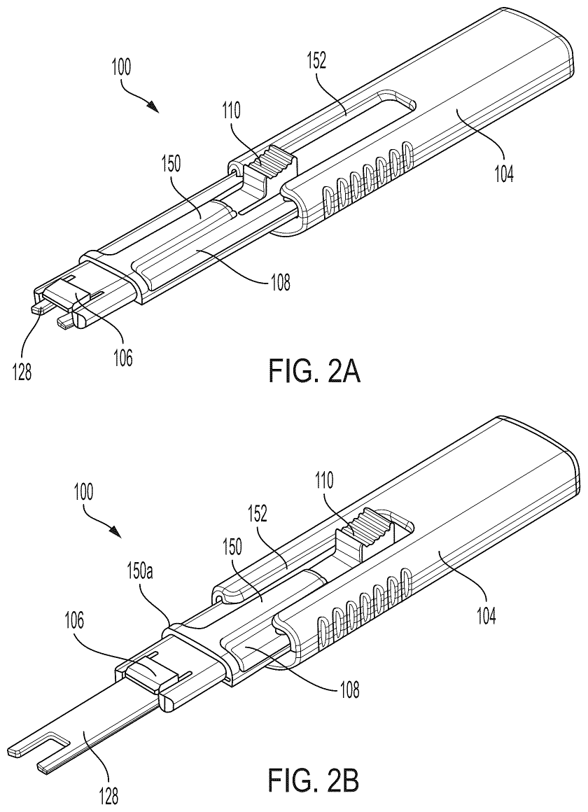

As noted previously, proximal movement of the elongated shaft 108, and an attached end effector 102, relative to a pusher 128 and handle 104 may cause the pusher 128 to extend into a cavity 116 of the end effector 102 containing a macroencapsulation device 400 disposed therein. FIGS. 2A-2B illustrate a portion of the implantation system 100 including the pusher 128, elongated shaft 108, and handle 102 to illustrate this relative movement. The end effector 102 is not illustrated for the sake of clarity. Specifically, FIG. 2A shows the elongated shaft 108 in the extended configuration and FIG. 2B shows the elongated shaft 108 in the retracted configuration where it has been moved proximally relative to the pusher 128. This would correspondingly move the end effector 102 in the proximal direction as well causing the pusher 128 to be displaced into the cavity of the end effector 102 to deploy the macroencapsulation device 400 as elaborated on further below.

During an operation the end effector 102 and distal portion of the elongated shaft 108 may be inserted into a surgical field of a patient. An actuation trigger 110 may then be actuated to permit relative movement of the elongated shaft and handle 104. For example, a depressible button that is configured to be depressed to unlock actuation of the implantation system 100 is illustrated in the figures. Once the actuation trigger 110 moved from a locked to an unlocked configuration, the trigger which is operatively connected to the elongated shaft may be moved proximally to move the elongated shaft 108 and the attached end effector 102, not depicted, proximally relative to the handle 104 and pusher 128. For example, a user's thumb may be used to depress the depicted button prior to displacing the button while the user grasps the handle 104. In either case, the depicted proximal movement of the trigger 110 causes the pusher 128 to extend out further from the distal end of the elongated shaft 108 into a channel and cavity of the end effector 102, described further below, which displaces the macroencapsulation device 400 out of the distal outlet 138 of the end effector 102 as the trigger 110, elongated shaft 108, and end effector 102 are moved in the proximal direction from the extended configuration towards the retracted configuration. As can be seen in FIG. 2B, when the implantation system 100 is in the retracted configuration, a portion of the elongated shaft 108 may be retracted into the handle 104.

To help guide appropriate movement of the elongated shaft in the desired proximal direction and to help avoid possible binding, it may be desirable to include one or more features configured to impose a desired movement of the elongated shaft 108 and trigger 110 relative to the handle 104. In some such embodiments, the handle 104 may include a slot that is sized and shaped to accommodate the predetermined movement of the actuation trigger 110 between the extended and retracted configuration. The slot may be an elongated linear slot formed in and extending along a portion of a length of the handle with an open distal end portion oriented towards the actuation trigger 110 in the initial fully extended configuration. A proximal end portion of the slot 152 may be positioned at a location corresponding to a position of the trigger 110 in the fully retracted configuration which may help limit excessive retraction of the implantation system 100 beyond the fully retracted configuration.

As noted above, in some embodiments, the slot 152 may include an open distal end portion. Correspondingly, the elongated shaft 108 may include a correspondingly sized and shaped protrusion 150 extending out from a surface of the elongated shaft. The protrusion 150 may be configured to be displaced into the slot 152 as the trigger 110 and elongated shaft 108 are displaced in the proximal direction relative to the handle 104. The protrusion 150 may include a portion that forms an elongated linear protrusion that forms a slip fit with the slot 152 in some embodiments. In either case, the protrusion 150 and slot 152 may cooperate to ensure the elongated shaft follows a desired movement path as the elongated shaft 108 is moved between the extended and retracted configurations. In some embodiments, the protrusion may also include a wider section 150a with a maximum transverse dimension that is larger than a maximum transverse dimension of the slot 152 which may again help to limit the retraction of the elongated shaft 108 into the handle 104 beyond a predetermined fully retracted configuration. For example, the protrusion 150 includes a pair of shoulders extending laterally out from a central portion of the protrusion 150 to form the wider section 150a that is wider than a corresponding width of the slot 152.

FIG. 3 illustrates the components of the implantation system in an exploded view. As can be seen in FIG. 3, the distal portion of the pusher 128 is elevated above the proximal portion. This allows for the narrower distal portion to protrude through the channel formed between the two portions of the elongated shaft 108a and 108b while the larger proximal portion is anchored to the handle portions 104a and 104b. The handle 104 may be split into a top portion 104a and a bottom portion 104b, wherein the top and bottom portions are positioned on opposing sides of the elongated shaft 108 and pusher 128. The elongated shaft 108 is split into a top portion 108a and a bottom portion 108b, wherein the top and bottom portions are connected to each other in a manner which creates a channel extending along a length of the elongated shaft. An actuation trigger 110 may be integrally formed with portion of the elongated shaft 108a as a cantilevered depressible button, though other trigger constructions may also be used. The trigger 110 may be used to transition an actuation lockout system between a locked and unlocked configuration to selectively prevent or permit relative displacement of the elongated shaft 108 and attached end effector 102 relative to the handle 104 and pusher 128 as elaborated on further below.

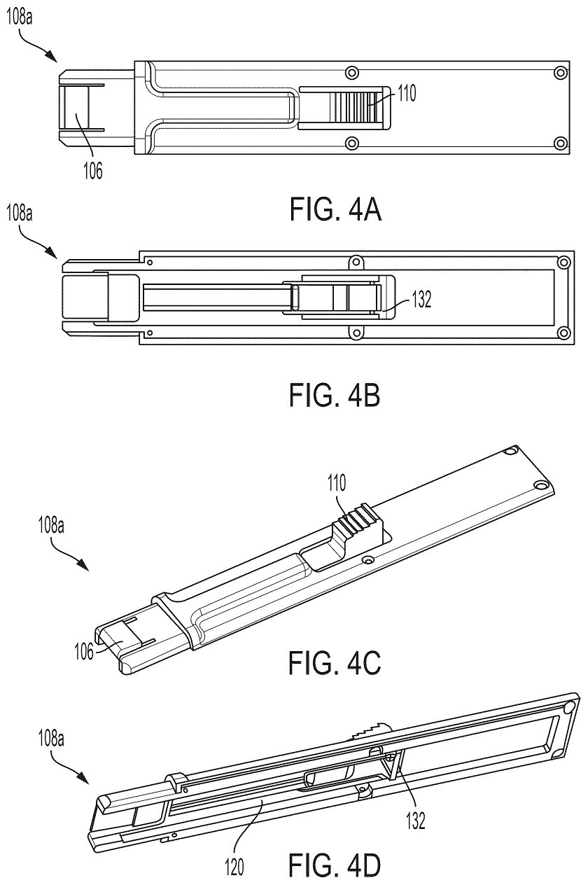

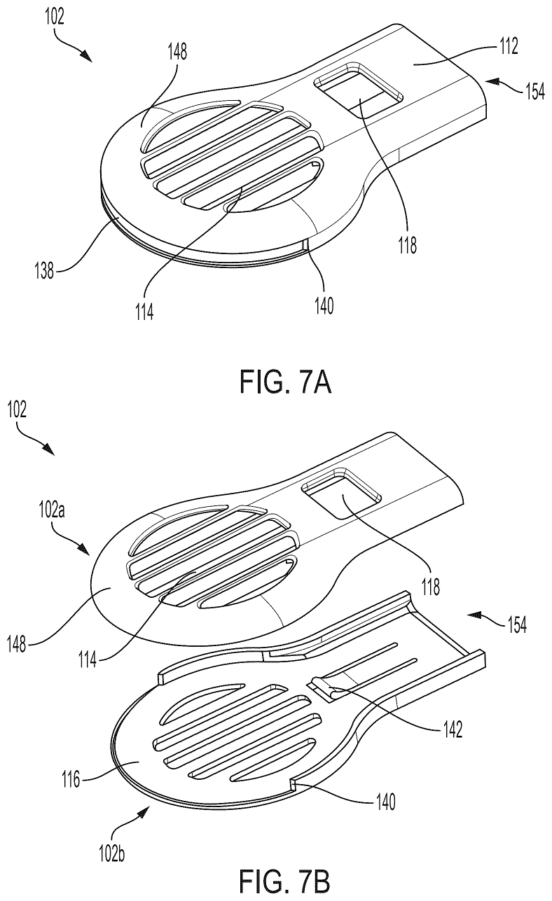

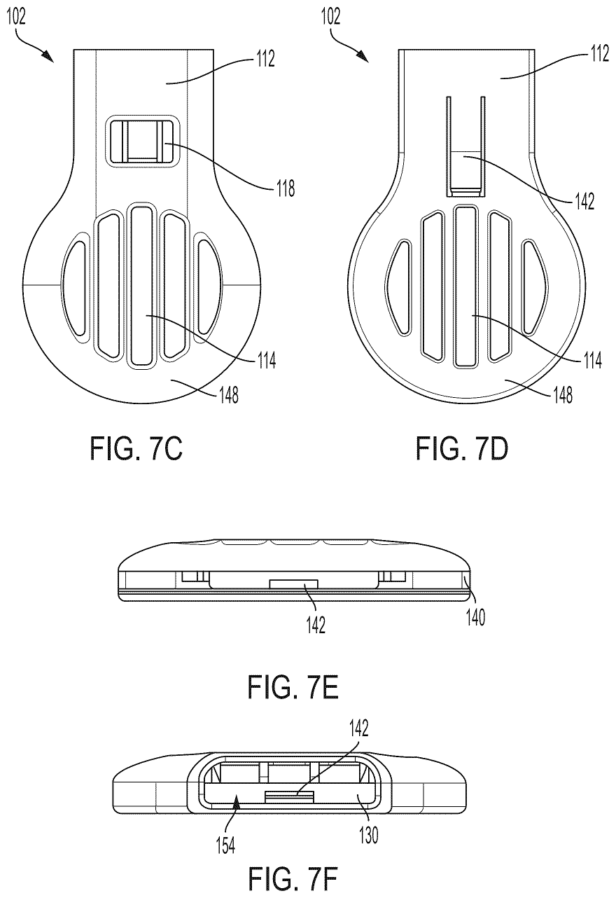

FIGS. 4A-4D depict the upper portion of the elongated shaft 408a including the trigger 110. The figures also illustrate one embodiment of the first portion of a connection 106. In this embodiment the first portion of the connection is a locking cam. As can further be seen in FIGS. 4A and 4B, the first portion of the connection 106 may be formed as a cantilevered catch connected to the elongated shaft 108 and extending in a distal direction. The cantilevered catch which is configured to engage a second portion of a connection 118, such as the opening seen in FIG. 7A.

FIG. 4D illustrates a portion of an internal channel 120 formed in the upper portion of the elongated shaft 102. The depicted channel 120 extends at least partially along a length and through a distal end portion of the elongated shaft 108 that the upper portion of the elongated shaft 108a forms. Again, the channel 120 may be sized and shaped to slidably receive a portion of a pusher 128 therein. In some embodiments, the internal channel 120 includes a distal opening oriented towards the end effector 102 such that the internal channel 120 may be aligned with an internal channel 130 and internal cavity 116 of the end effector 102 when the end effector 102 and elongated shaft 108 are connected as detailed further below.

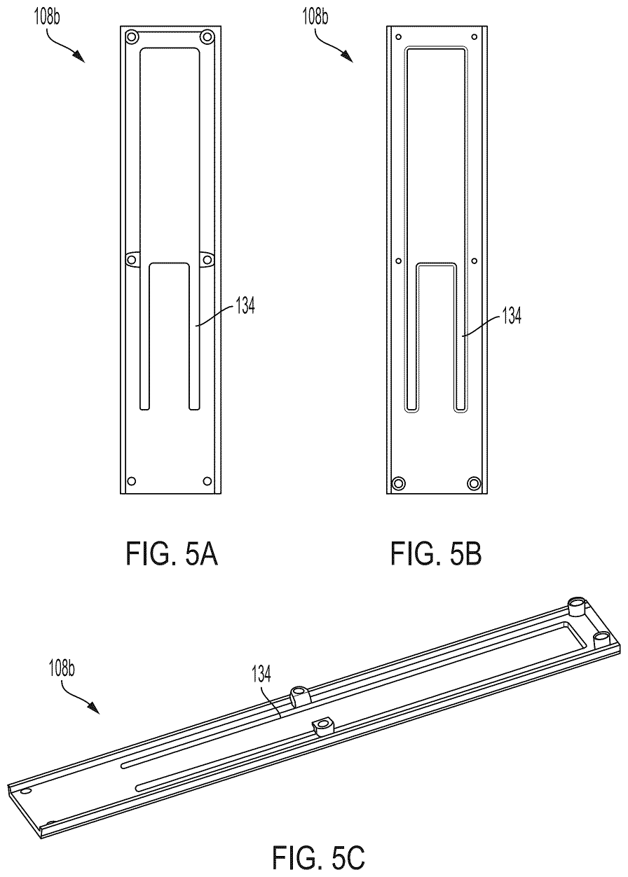

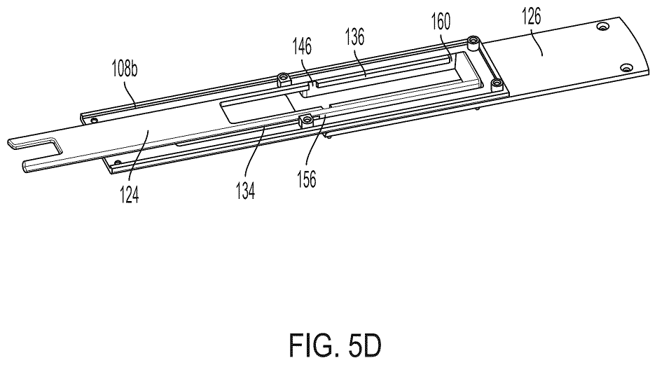

FIGS. 5A-5C depict a bottom portion of the elongated shaft 108b. The bottom portion of the elongated shaft may be configured to be connected to the top portion of the elongated shaft 108a using any appropriate connection, see FIGS. 4A-4D. A pusher shaft 124 may be slidably disposed between the two opposing portions of the elongated shaft 108. The top portion of the elongated shaft 108a and the bottom portion of the elongated shaft 108b may be connected in various ways. Although currently depicted with threaded fasteners having corresponding holes in the top portion 108a and threaded holes formed in the bottom portion 108b, the various portions of the elongated shaft 108 may be connected via adhesives, welds, interlocking mechanical features or any other connection suitable for connecting the top and bottom of the elongated shaft.

The bottom portion of the elongated shaft 108b may be relatively flat and may extend along a length of the upper portion of the elongated shaft 108a. Thus, a distal portion of the pusher 128 may extend through the channel 120 formed between the upper 108a and lower 108b portions of the elongated shaft. Additionally, the bottom portion of the elongated shaft 108b may be constructed with a hole in a proximal portion of the shaft which allows a proximal portion of the pusher 128 to be connected to the distal portion of the pusher 128 through the opening formed in the bottom portion of the elongated shaft 134.

FIG. 5D depicts the bottom portion of the elongated shaft 108b assembled with the pusher 128 with a distal pusher shaft 124 of the pusher 128 extending from a base 126 of the pusher 128 through an opening formed in the bottom portion of the elongated shaft 108b. As mentioned above, in this embodiment, the bottom portion of the elongated shaft 108b is slidably connected to the pusher 128. To help appropriately guide and limit the motion of the pusher 128 relative to the elongated shaft 108, the elongated shaft 108, in the depicted embodiment the bottom portion of the elongated shaft 108b, may include one or more guides 134 formed therein that are configured to slidably receive a wall 156 extending between the pusher shaft 124 and pusher base 126 located on opposing sides of the opening formed in the bottom portion of the elongated shaft 108b. In the depicted embodiment, the guides 134 formed on opposing sides of a central longitudinal axis of the elongated shaft are linear slots which are sized and shaped to receive corresponding opposing walls of the pusher 128. This permits the pusher 128 and elongated shaft 108 to move longitudinally relative to each other until the one or more walls 156 come into contact with an end portion of the associated one or more guides. Thus, the guides 134 and slidably received walls 156, or other appropriate interlocking portions of the pusher 128, may help to limit a range of motion of the elongated shaft 108 and pusher 128 to be within a predetermined range corresponding to the fully extended and fully retracted configurations while also helping to ensure the motion is parallel to a longitudinal axis of the system.

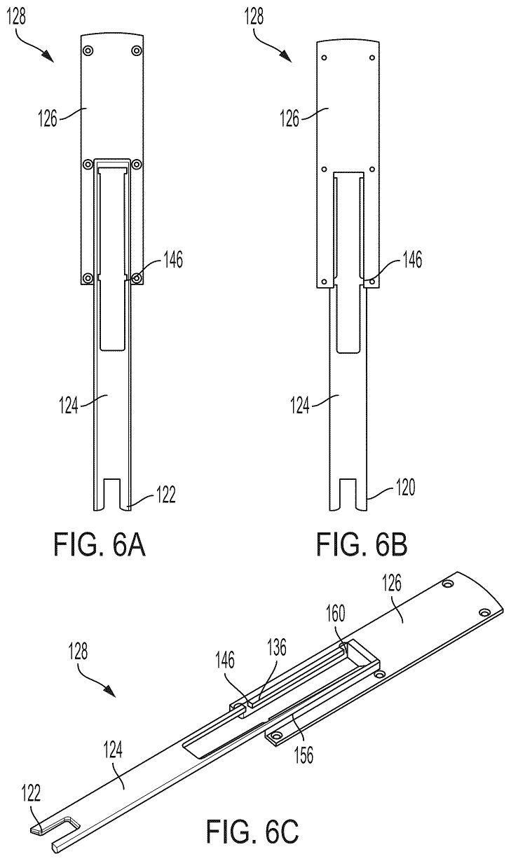

FIGS. 6A-6C depict one embodiment of a pusher 128. As mentioned above, the pusher 128 may comprise at least two portions. The first portion, a pusher shaft 124, is configured to be slidably positioned within the internal channel 120 of the elongated shaft. The distal tip of the pusher shaft is a pushing surface 122 which is configured to extend through the internal channel 130 of the end effector to contact the macroencapsulation device 400 when the elongated shaft is in the retracted position. The second portion, a pusher base 126, is configured to anchor the pusher 128 as a whole to the handle and may not be disposed within the internal channel 120 of the elongated shaft. As can be seen in any of FIGS. 6A-6C, the pusher shaft 124 is narrower and extends distally from the pusher base. This compliments the above noted benefits associated with the elongated shaft having a relatively small transverse dimension. Specifically, having a narrow, elongated shaft increases the maneuverability of the implantation system within a patient. In some embodiments, the pushing surface 122 includes two separate portions corresponding to two separate protrusions extending distally from a distal end portion of the pusher 128 and on opposing sides of a longitudinal axis of the pusher shaft 124. The protrusions may be configured to contact a proximal portion of a frame of the macroencapsulation device 400. The pushing surface may also be sized and shaped to compliment a size and shape of a frame of the macroencapsulation device 400. For example the pushing surface 122 may also be vertically and horizontally curved to at least partially conform to the outer surface of a frame of the macroencapsulation device 400. These features may assist the pusher 128 in gently maintaining the position of the macroencapsulation device 400 while the end effector 102 is retracted.

The first portion of the actuation lock 132, illustrated as a protrusion, is seen in FIG. 4B and FIG. 4D. The first portion of the actuation lock 132 is connected to the actuation trigger 110, which causes the first portion of the actuation lock 132 to move when force is applied to the actuation trigger 110. Moving the first portion of the actuation lock 132 may displace the first portion of the actuation lock 132 from a locked configuration adjacent to the second blocking portion of the actuation lock 136 to unlock the system. In other words, depressing the actuation trigger 110 moves the first portion of the actuation lock 132, which in some embodiments is a protrusion connected to the actuation trigger 110, out of alignment with the second blocking portion of the actuation lock 136 form on the pusher 128. This transitions the actuation lock from a locked configuration, where movement of the actuation trigger is prevented, to an unlocked configuration where the actuation trigger 110 and associated elongated shaft 108 are able to move in a direction parallel to a longitudinal axis of the system relative to the pusher 128 and handle 104. For example, in the depicted embodiment, the illustrated protrusion may initially be located within a gap between two aligned rails that prevent movement of the actuation trigger 110 in the proximal and distal directions. Actuation of the trigger 110 may displace the protrusion out of the gap into an unlocked configuration where the protrusion, and the overall trigger, may be free to move in a direction parallel to a longitudinal axis of the implantation system 100. Regardless of the specific configuration, when in the unlocked configuration, the first portion of the actuation lock 132 (i.e., the protrusion) may slide along the rails of the second blocking portion of the lock 136 or other appropriate portion of the system permitting actuation of the system. In some embodiments, the system may include a second locked configuration after the elongated shaft 108 is fully retracted. The depicted actuation lockout system may help to prevent inadvertent and/or partial deployment of the macroencapsulation device. The first portion of the actuation lock 132 may also prevent the elongated shaft 108 from returning to the extended position once retracted. For example, a second gap 160 may be sized and shaped to accept the first portion of the actuation lock 132 therein to prevent movement of the actuation trigger 110 when fully retracted. In the specifically depicted embodiment, the protrusion present on the depressible button may cam upwards into the second gap 160 after the protrusion has slid across the illustrated rails during actuation which may prevent extension of the elongated shaft 108 from the retracted configuration towards the extended configuration due to the rails blocking movement of the elongated shaft in the distal direction.

FIGS. 7A-7F depict one embodiment of an end effector 102 that may be connected to an elongated shaft of an implantation system. As described previously, the end effector 102 may include a neck portion 112 that extends proximally from a wider body portion 148. Conversely, the body portion 148 may be attached to and extended distally out from a distal end portion of the neck portion 112. The neck portion 112 may be configured to be attached to a distal end portion of an elongated shaft 108 of an implantation system. As mentioned above, this attachment may be releasable or may be permanent. As illustrated in the figure, a maximum transverse dimension of the neck portion 112 in a direction perpendicular to a longitudinal axis of the end effector 102 may be less than a maximum transverse dimension of the body 148 in the direction perpendicular to a longitudinal axis of the end effector 102. Correspondingly, the maximum transverse dimension of an elongated shaft 108 that is configured to be attached or is integrally formed with the end effector 102 may have a maximum transverse dimension that is less than a maximum transverse dimension of the body 148. The elongated shaft 108 may also have a maximum transverse dimension that is equal to or less than a maximum transverse dimension of the neck 112.

In the depicted embodiment, the end effector 102 is formed from an upper portion of the end effector 102a that is connected to an opposing lower portion of the end effector 102b. The end effector 102 may include a cavity 116 formed in an interior of the body. The cavity 116 may be sized and shaped to receive the macroencapsulation device 400 therein.

In some embodiments, at least a portion of a cross section of the cavity 116 in a plane parallel to the longitudinal axis of the end effector is round. For example, the depicted cavity 116 and body 148 of the end effector 102 form a semi-circular shape with the neck 112 extending proximally from the semi-circular body. Given these, and other possible shapes of the body and cavity, a maximum transverse dimension of the cavity 116 may be greater than a maximum transverse dimension of both the channel extending through the elongated shaft 108 and a corresponding aligned channel formed in the neck 112 of the end effector 102.

As shown in the figures, a first opening 154 may be formed on a proximal end portion of the neck 112 such that the opening 154 is oriented towards the elongated shaft when connected thereto. The opening 154 may be sized and shaped to receive a portion of the elongated shaft therein such that an internal channel 120 of the elongated shaft, see FIGS. 3-5D, and an internal channel 130 of the end effector 102 may be aligned in this connected configuration. Accordingly, a pusher 128 extending out from the elongated shaft 108 may extend into internal channel 130 of the end effector 102 when the end effector 102 is connected to the elongated shaft 108. An outlet 138 may be formed on a distal portion of the body 148. The distally oriented outlet 138 may be sized and shaped to permit the macroencapsulation device 400 to be displaced out of the cavity 116 through the outlet 138. The outlet 138 may be connected to the cavity 116 formed within the body portion 148 which is configured to contain the macroencapsulation device 400 therein prior to deployment. As illustrated in the depicted embodiment, a maximum transverse dimension of the outlet 138 may be equal to or greater than a maximum transverse dimension of the cavity 116.

An opening formed in a side portion of the neck 112 of the end effector 102 may be used to form a second portion of a connection 118 which is configured to interlock with with the first portion of the connection 106 formed on the elongated shaft 108. As mentioned above, the first portion of the connection 106 may be a cantilevered hook which is configured to be cammed into the opening forming the second portion of the connection 118. In order to remove the end effector from the elongated shaft, the first portion of the connection 106 may be depressed below an inner surface of the second portion of the connection 118 permitting the two components to be moved apart.

A plurality of slots 114 may be formed on the body portion 148 of the end effector 102, wherein a long axis of each slot of the plurality of slots is aligned with the longitudinal axis of the implantation system 100 and end effector 102. The slots 114 may permit the diffusion of media into and out of the end effector 102 and a macroencapsulation device 400 stored within the cavity 116 of the end effector 102. The macroencapsulation device 400 benefits from access to media which may include nutrients and oxygen as the macroencapsulation device 400 may house one or more populations of cells disposed therein. Additionally, the orientation of the slots 114 may be aligned with a direction of insertion into a patient and/or relative movement between the components of the implantation system. This may assist in minimizing abrasive forces to both the patient and the macroencapsulation device 400 while the implantation system is being used.

In addition to the above noted features, as beast seen in FIGS. 7A and 7B, an end effector 102 may include a pair of opposing ledges 140 disposed on opposing sides of the distally oriented outlet 138 and may extend between opposing upper and lower portions of the end effector to form the cavity 116 therebetween. In such an arrangement, the ledges may be formed by opposing side walls of the end effector extending between the upper and lower portions of the end effector 102. In some embodiments, the two opposing ledges 140 may be located proximally from a distal most portion of the end effector 102 and may form a proximal most portion of the outlet 138. Accordingly, the opposing ledges 140 may be configured to interact with one or more other corresponding features formed on a corresponding support or other structure to assist in holding the end effector in a desired pose as elaborated on further below in regard to the containers and supports disclosed herein.

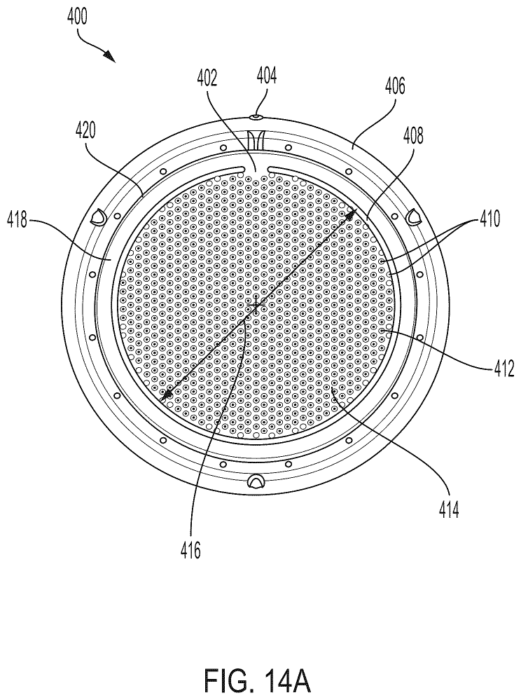

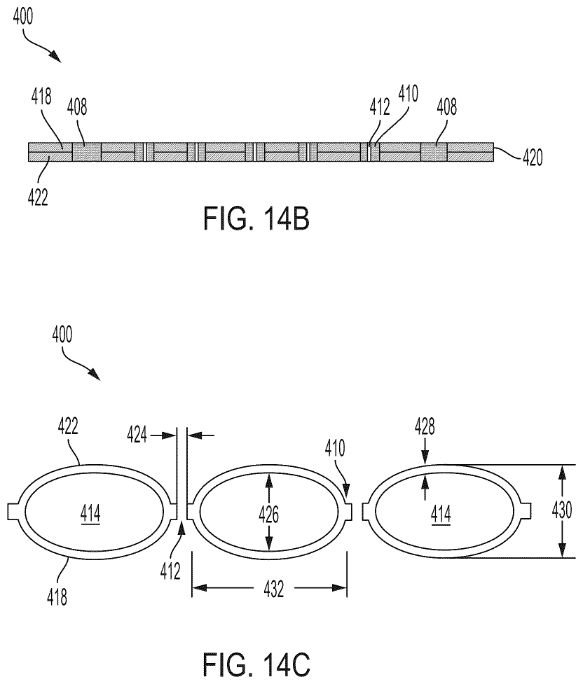

Due to storing and transporting a macroencapsulation device 400 within an end effector 102, it may be desirable to apply a retaining force to maintain the macroencapsulation device 400 in a desired pose within the end effector 102 to prevent an inadvertent or partial deployment of the device. Therefore, in some embodiments, an end effector 102 may include one or more retaining tabs 142 configured to apply a retaining force to a frame 406 of the macroencapsulation device 400, see FIG. 14A. Although only one retaining tab is depicted in this embodiment, it should be understood that multiple retaining tabs are contemplated. The retaining tab 142 is oriented in a distal direction and extends from the neck portion 112 into the cavity 116, where the microencapsulation device 400 is configured to be disposed. The retaining tab 142 may assist in ensuring that the macroencapsulation device 400 remains in the correct pose and position within the end effector 102. The retaining tab 142 may be configured to be overcome by the force of the pusher 128 on the macroencapsulation device 400 during deployment. The retaining tab 142 is depicted with a cantilevered structure. The retaining tab may be hooked, or flat, so long as the retaining tab contacts the macroencapsulation device with a retaining force sufficient to avoid the device being displaced distally out of the cavity 116 without the pusher applying a force greater than the retaining force to the frame of the macroencapsulation device 400. The system may benefit from the retaining tab contacting the frame 406 of the macroencapsulation device 400. This orientation assists in minimizing the contact to the one or more membrane layers of the macroencapsulation device 400 in order to minimize and avoid damage to the fragile membrane layers.

As best shown in FIGS. 7E-7F, an end effector may include a keyed transverse cross-sectional shape. In such an embodiment, one or more portions of the end effector 102 may be non-symmetric to help ensure a desired orientation of the end effector 102 when interacting with one or more other components or systems as elaborated on further below. Specifically, the front and back views depict the lack of symmetry in the shape of the device. In this embodiment one portion of the end effector 102 has a transverse cross section with flat profile whereas another portion of the end effector has transverse cross section with a non-linear profile. In the depicted embodiment, the non-linear portion of the profile is a curved profile. Of course, while a particular keyed profile has been depicted, it should be understood that end effectors with different cross-sectional shapes are also contemplated.

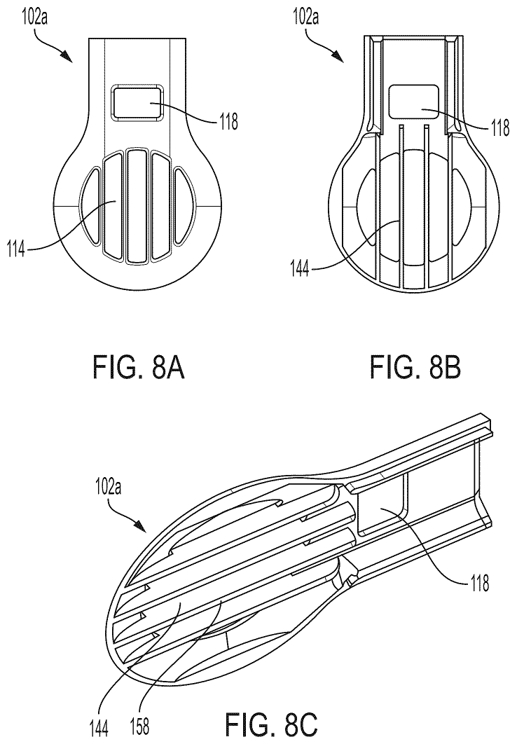

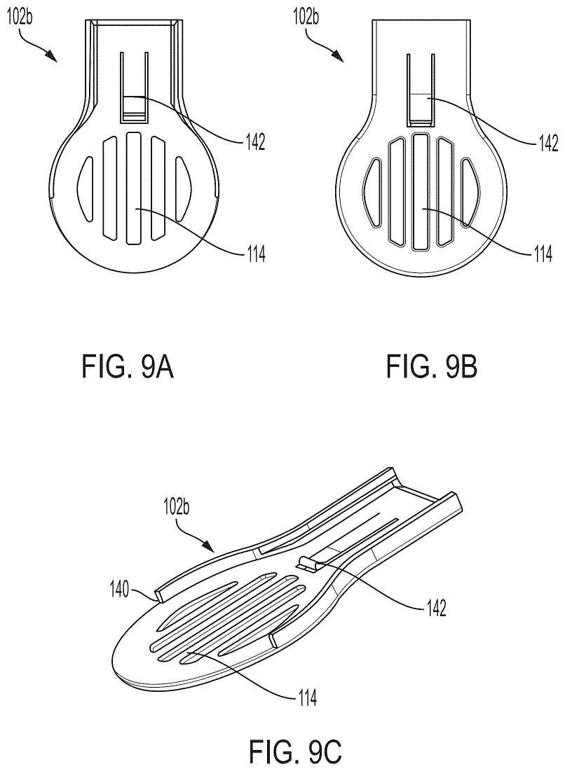

FIGS. 8A-8C depict the top portion of the end effector 102a. FIGS. 9A-9C depict perspective views of the bottom portion of the end effector 102b. Many of the features depicted in FIGS. 8A-9C have been discussed in the disclosure related to FIGS. 7A and 7B. However, FIG. 8C also illustrates the fins 144 disposed on the interior surface of the end effector body 148 and between the slots 114. The fins 144 may assist the end effector 102 in securely storing the macroencapsulation device 400 by removing at least a portion of the dead space in the cavity 116 when an end effector includes a transverse cross section with a curved or other non-linear profile. The fins 144 may also assist in positioning the macroencapsulation device 400 adjacent to the outlet 138. Although a solid structure with slots could be used, the fins 144 may improve both the manufacturing process and the operation of the end effector. The fins 144 are connected to the inner surface of the top portion of the end effector 102a. The height of the fins 144 may vary in both the longitudinal direction and the transverse direction. This is due to the curvature of the top portion of the end effector 102a varying in both width and length directions. The fins 144 extend from an exterior portion of the body inwards towards the cavity 116. The lower surface of the fins 158 oriented towards the cavity 116 may be positioned within a plane which forms the upper surface of the cavity 116. Accordingly, the lower surface of the fins 158 may create a set of flat surfaces disposed within a common plane which contact the macroencapsulation device 400, when it is disposed therein, to both maintain the macroencapsulation device 400 in a desired location within the end effector while also guiding the macroencapsulation device 400 during deployment from the end effector 102. Although there are four fins depicted in the figures, it should be understood that any reasonable number of fins may be present. The fins may be oriented parallel to both the relative direction of movement of the different components as well as a longitudinal axis of the system. This may help to avoid unnecessary abrasion applied to the macroencapsulation device 400 during insertion and implantation.

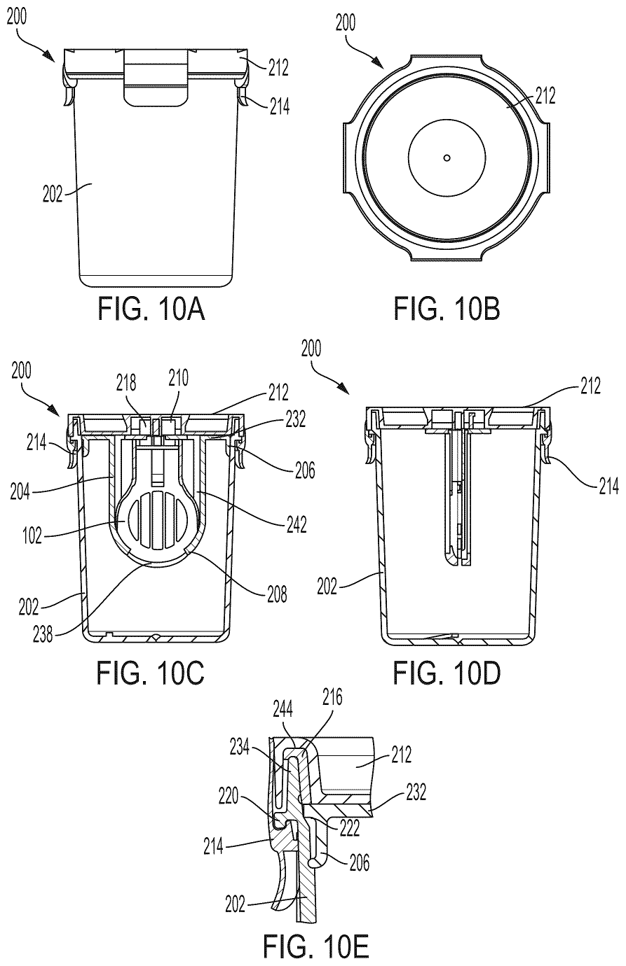

FIGS. 10A-10E depict one embodiment of a macroencapsulation device storage system 200 with a lid 212 disposed thereon. The macroencapsulation device storage system 200 includes a container 202, a lid 212, and a support 204 configured to maintain an end effector 102 in a desired pose within the container 202. For example, the support 204 and container 202 may be configured to maintain the end effector 102 in a desired pose within the container 202 such that the end effector 102 may be maintained immersed in a liquid medium within the container 202 including, for example, a cell culture or cell storage media. For example, in some embodiments, the device storage system comprises a liquid medium comprising Dulbecco's Modified Eagle Medium (DMEM), Ham's F-12 nutrient mix (F-12), and human serum albumin (HSA). In some embodiments, the medium comprises between about 0.25% and 2.5% HSA. Accordingly, in some embodiments, the medium comprises about 0.25% HSA. In some embodiments, the medium comprises about 0.5% HSA. In some embodiments, the medium comprises about 0.75% HSA. In some embodiments, the medium comprises about 1% HSA. In some embodiments, the medium comprises about 1.25% HSA. In some embodiments, the medium comprises about 1.5% HSA. In some embodiments, the medium comprises about 1 0.75% HSA. In some embodiments, the medium comprises about 2% HSA. In some embodiments, the medium comprises about 2.25% HSA. In some embodiments, the medium comprises 2.5% HSA. As elaborated on further below, this may help to protect a macroencapsulation device 400, not shown, disposed in the end effector 102 from damage while also maintaining a viability of one or more populations of cells disposed in the macroencapsulation device.

In some embodiments, the container 202 includes an interior volume and an opening 236 in fluid communication with the interior volume. As shown in the figures, the container 202 may have an approximately cylindrical exterior shape with an opening 236 with a transverse cross section that is approximately equal in size and shape to a transverse cross section of the interior volume of the container 202. However, containers 202 and openings 236 exhibiting different combinations of sizes and/or shapes are also contemplated. A lip 234 may extend around a perimeter of the opening 236 where the lip 234 extends out from the container 202 in a direction oriented at least partially away from the opening. The container 202, opening 236, and lip 234 are depicted as having circular transverse cross-sectional shapes. However, different cross-sectional shapes may also be used as the disclosure is not so limited.

A lid 212 may be correspondingly sized and shaped such that the lid 212 covers the opening 236 when assembled onto the container 202. The lid 212 may be configured to be selectively coupled to the opening of the container 202 by any appropriate connection. For example, as shown in the figures, a first portion of a connection 214 formed on the lid 214 may be configured to be connected to a second portion of the connection 220 formed on the container 202 when the lid 212 is in the closed configuration. This connection may either be a reusable connection where the first and second portions may be repeatably connected and disconnected or the connection may be a single use connection where the first and second portions may be permanently disconnected after a single use. In the depicted embodiment, the first portions of the connection 214 are illustrated as a plurality of rotatable tabs distributed around a perimeter of the lid 212. Each tab includes a catch that is rotatably attached to the lid 212 via a living hinge or other appropriate rotatable connection (e.g., pin joints, hinges, etc.). The second portions of the connection 220 illustrated in the figures is a correspondingly shaped shelf or other structure including a divot, groove, recess, or other appropriately sized and shaped feature configured to be engaged with and retain a catch of an associated tab in the locked configuration. When it is desired to move the lid to the unlocked configuration, the plurality of tabs corresponding to the first portion of the connection 214 may be rotated outwards away from the container 202 to place the lid 212 in an unlocked configuration. Once unlocked, the lid 212 may be removed from the opening 236 of the container 202. Correspondingly, in some embodiments, the tabs may be rotated inwards towards the container to lock, or relock, the opposing first and second portions of the connection 214 and 220 to attach the lid 212 to the container 202.

In the above embodiment, the use of rotatable locking tabs is described. However, it should be understood that the current disclosure is not limited to only being used with the illustrated type of connection. For example, the first and second connections may be configured in any appropriate manner to permit a user to selectively unlock the lid 212 from the container 202. Exemplary types of connections may include, but are not limited to, a pull tab connection, clamps, latches, interference fits, threaded fasteners, adhesives exhibiting sufficiently low peel strengths, and/or any other appropriate type of releasable connection configured to permit a lid 212 to be selectively removed from an opening 236 of a container. It is noted that while a threaded connection could be used to couple the lid 212 to the container 202, the size of the container 202 and lid 212 may result in undesirably large opening forces. Accordingly, the disclosed rotatable tabs may offer a desired combination of compression strength and ease of operation.

During use of other container systems, it was noted by users that containers including typical threaded connections combined with typical seals led to undesirable binding of the lid on the container in some instances. Accordingly, as noted previously, the use of a wedge seal in combination with the disclosed connections used with a macroencapsulation device storage system 200 may desirably provide a system that is biased towards an open configuration when unlocked while also providing a robust seal to avoid contamination and/or leakage.

FIG. 10E depicts a close-up view of the connection and seal of the container 202 and lid 212. Again the lip 234 extends outwards from the opening 236 of the container 202. The lid 212 includes a correspondingly sized and shaped groove 244 extending around a perimeter of the lid 212 that is configured to at least partially receive the lip 234 disposed therein when the lid 212 is positioned on and covering the opening 236 of the container 202. An elastic seal 216 may be disposed in and extend along at least a portion, and in some embodiments, an entire length of the groove 244. During insertion of the lip 234 into the groove 244, the lip 234 is brought into contact with and is compressed against the elastic seal 216 to form a seal therebetween. Thus, a seal may be formed around the opening 236 to isolate the interior volume of the container 202 from the surrounding exterior environment when the lid 212 is disposed on the container 202. As noted above, the first and second portions of a connection 214 and 220 may maintain the elastic seal 216 and lip 234 compressed against each other in the closed configuration when the first and second portions of a connection 214 and 220 are locked together.

To aid in biasing the lid 212 towards an open configuration without binding, in some embodiments, the elastic seal 216 and lip 234 may be configured to form a wedge seal. In the depicted embodiment, the elastic seal 216 conforms to a shape of a portion of the groove 244 the elastic seal 216 is disposed in. The elastic seal 216 includes a channel extending along a length of the elastic seal 216 that is sized and shaped to receive a portion of the lip 234 therein. A base of the lip 234 may be wider than a tip of the lip 234 inserted into the channel of the elastic seal 216. Correspondingly, one or both of the opposing interior and exterior oriented surfaces of the lip 234 may be angled relative to a longitudinal axis of the container extending through the opening. In some embodiments, the longitudinal axis may be a vertical axis parallel to a direction of gravity when a base of the container 202 is disposed on a level supporting surface. Correspondingly, the elastic seal 216 may form one or more angled interfaces with the lip 234 when the elastic seal 216 and lip 234 are compressed together. These one or more angled interfaces may form angles relative to the longitudinal axis that are within the ranges previously noted above. Additionally, at least a component of a direction normal to the interface and extending away from the lip 234 may extend vertically upwards out of the opening 236. In embodiments in which two opposing interfaces on an interior and exterior side of the lip 234 are formed with the elastic seal 216, a magnitude of the angle between the interfaces and the longitudinal axis of the container 202 may be different. For example, as shown in FIG. 10E, a magnitude of the angle between the longitudinal axis and the interface between the lip 234 and the elastic seal on the interior side is greater than on the exterior side of the lip 234. Additionally, as shown in the figure, a thickness of the elastic seal 216 may be different adjacent to the opposing interior and exterior sides of the lip 234 when assembled together. For instance, a thickness of a portion of the elastic seal 216 configured to be placed in contact with the interior surface of the lip 234 may be thicker than a portion of the elastic seal 216 configured to be placed in contact with the exterior surface of the lip 234 when the lid 212 is connected to the container 202.

The one or more angled interfaces formed between the lip 234 and the elastic seal 216 in the closed configuration may offer several benefits. For example, the disclosed wedge seal may offer better sealing due to both improved compression due to mechanical advantage using the wedge seal and larger interfacial areas. For instance, the seal formed between the elastic seal 216 and the lip 234 may extend from an interior surface around a tip portion to an exterior surface of the lip 234 with the lip 234 compressed between two opposing portions of the elastic seal. Additionally, when the lip 216 and elastic seal 234 are compressed together when the lid 212 is held in the closed configuration by the associated first and second portions of the connection 214 and 220, the resulting force applied at the one or more angled interfaces between the lip 234 and the elastic seal 216 may bias the lid 212 towards the open configuration. Thus, when the compressive force applied to maintain the lid 212 in the closed configuration with the container 202 is released by opening the associated first and second potions of the connection 214 and 220, the lid 212 may be automatically biased towards the open configuration without binding which may facilitate a user removing the lid 212 from the container 202.

It should be understood that the elastic seals described relative to any of the embodiments disclosed herein may be made from any suitable elastic material exhibiting sufficient elasticity to permit the lip 234 and elastic seal 216 to be compressed together to form a seal. Appropriate materials may include elastic polymers, elastomers, and/or other sufficiently elastic materials. Specific examples of appropriate materials may include but are not limited to silicone rubbers, thermoplastic elastomers, ethylene propylene diene monomer (EPDM), polytetrafluoroethylene (PTFE), Nitrile, synthetic rubber polymer or Viton. Of course, it should be understood that the disclosed elastic seals are not limited to only these specific materials.

As noted previously above, in some embodiments, a support 204 is configured to support an end effector 102 containing a macroencapsulation device 400 disposed in the end effector 102 in the container 202 in a predetermined pose. Maintaining the end effector 102 in a predetermined pose within an interior volume of the container 202 may assist in maintaining the macroencapsulation device 400 immersed in a desired media disposed in the container 202 and may help to prevent damage to the macroencapsulation device 400 during storage, transport, and use.

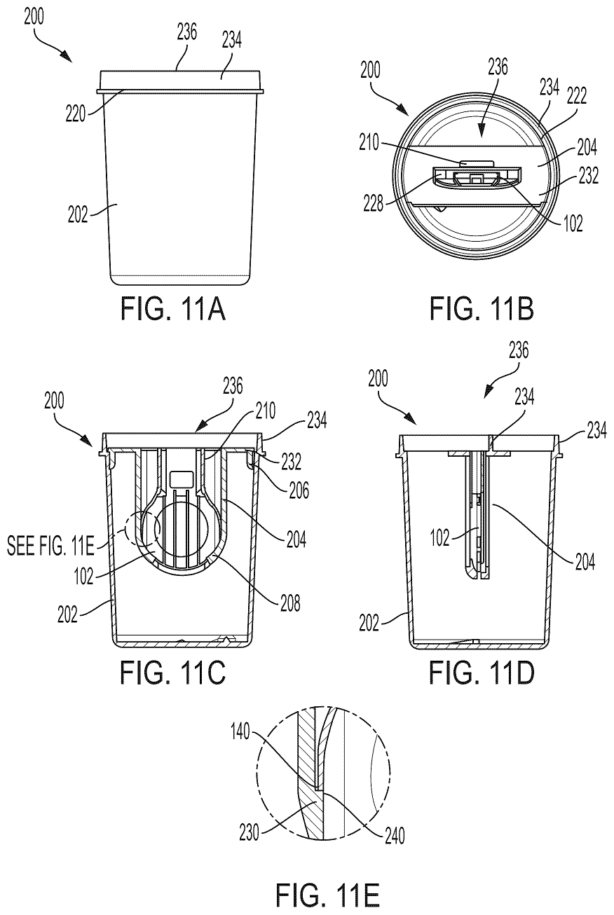

As best seen in FIGS. 11A-11E which depict the macroencapsulation device storage system 200 in the open configuration with the lid 212 removed, as well as FIG. 10E, the support 204 is configured to be supported in a predetermined pose within an interior volume of the container 202. The support 204 includes a base 232 that is configured to extend across at least a portion of a width of the interior volume of the container. For example, in some embodiments, the base 232 may be an elongated plate though other form factors may also be used. The base 232 includes one or more portions that are configured to be disposed on one or more supporting features such as the illustrated supporting ledge 222 of the container 202. In the depicted embodiment, the supporting ledge 222 is sized and shaped such that a corresponding portion of the base 232 may be disposed thereon when the support 204 is disposed in the container 202 to maintain the desired pose of the support 204. The support may include one or more tabs 206 extending from a bottom surface of the base 232 towards an interior of the container 202 when the support 204 is disposed therein. In the depicted embodiment, the support 204 includes two tabs 206 disposed on opposing end portions of the base 232 which include hooks oriented radially outwards from a central longitudinal axis of the support 204. In some embodiments, the one or more tabs may be compressed against an interior surface of the container when the support 204 is disposed therein which may help to maintain a position of the support 204 within the container 202. Alternatively or additionally, the lid 212 may also help to maintain a pose of the support 204 within the container. Specifically, in some embodiments, the lid 212 my compress the base 232 of the support 204 against the supporting ledge 222 of the container 202 when the lid is held in the closed configuration which may restrict the movement of the support 204 within the interior volume of the container 202.

In addition to the above, to help facilitate handling of a support during insertion and removal from a container, in some embodiments, a support 204 may include a handle 210 disposed on and extending out from an upper surface of the base 232 that is oriented outwards relative to the opening 236 when the support 204 is disposed in the container 202. In the depicted embodiment, the handle extends out from the base and may be configured to be grasped and manipulated by a tool, robotic manipulator, and/or hand of a user depending on the application and thus may correspond to any number of different appropriate configurations.

As noted previously, an end effector may have a keyed shaped. Accordingly, and as best seen in FIG. 11B, a support 204 may include a correspondingly sized and shaped opening 228 formed on the base 232. To facilitate the insertion of an end effector 102 in a desired pose relative to the support 204 and container 202, the opening 228 of the support 204 may be a keyed opening having a size and shape that conforms to a size and shape of the end effector 102 that is inserted through the opening 228. Specifically, the opening 228 and the end effector may have transverse cross sections with shapes that include at least one degree of asymmetry. Therefore, an end effector 102 may only be inserted into the support 204 in a single orientation which may help to enforce the desired pose of the end effector in the support 204. For example, the cross sectional shapes may include flat and curved portions as discussed previously above.

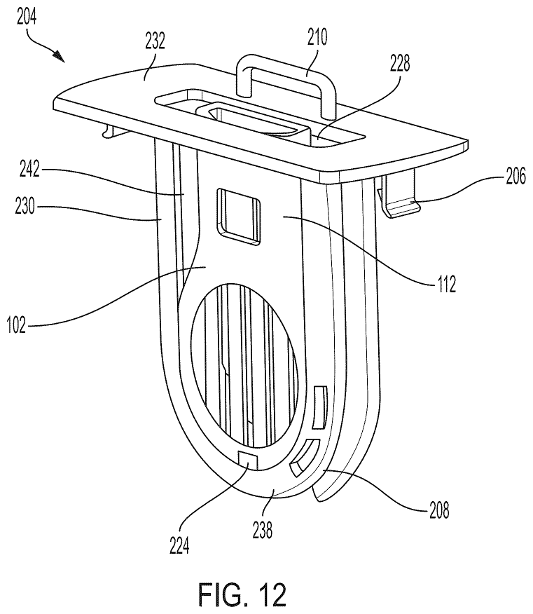

FIG. 11E depicts a close-up view of the end effector supporting ledge 240 seen in FIG. 12C. In the depicted embodiment, a supporting ledge extends inwards towards a central longitudinal axis of the support from one or both rails 230 of the support 204. The supporting ledge 240 is configured to maintain a position of the end effector in the support 204. Specifically, as noted above, the end effector 102 may include one or more corresponding ledges 140 formed on opposing sides of the outlet 138, see FIGS. 7A-7B. The one or more ledges 140 of the end effector 102 may be configured to be disposed on the one or more ledges 240 oriented in a direction directed at least partially out of an internal volume of the container 202 and formed on one or more corresponding portions of the support 204. This may help to maintain a desired position of the end effector 102 relative to a direction of insertion of the end effector 102 into the support 204. Thus, as mentioned above, in some embodiments, the one or more support ledges 240 assist in storing a macroencapsulation device 400 in a desired pose within an internal volume of the container 202 prior to closing the container 202 with a corresponding lid 212 as described above.

FIG. 12 depicts the support 204 of the above embodiments in isolation for the sake of clarity. As shown in the figure, the end effector 102 has been inserted through the opening 228 extending through the base 232. A pair of rails 230 positioned on opposing sides of the opening 228 extending away from the base 232. Each rail includes a corresponding groove 242 formed therein and extending along at least a portion of a length of the rail. The grooves 242 are configured to capture a corresponding portion of the end effector 102 located within the grooves 242 to maintain the end effector 102 and the macroencapsulation device 400 disposed in the support 204 between the rails 230 and within the opening 228. The rails 230 and grooves 242 may include a distal portion that spaced apart from the base 232 that is shaped to conform to a shape of a distal portion of the end effector 102. For example, the rails 230 and grooves 242 include curved end portions that are curved inwards relative to a central longitudinal axis of the support 204 which may prevent the end effector 102 from being displaced out from between the rails 230.

In some embodiments, the pair of rails 230 may be connected by a segment 238 extending between the rails 230. The segment 238 may be curved, and in some instances, may conform with a curvature of the adjacent end portions of the rails 230 and the end effector 102 disposed therebetween. However, instances in which a differently shaped segment extends between the rails 230 are also contemplated. While the rails 230 may help to maintain a desired pose of an end effector 102 within a support 204, in embodiments where the end effector 102 does not include a uniform length or increasing length in the proximal direction may result in it being difficult to maintain a desired pose of the end effector 102. Specifically, the neck 112 of the end effector 102 is narrower than the spacing between the rails 230 which may permit the end effector 102 to rotate between the rails 230.

To help mitigate movement of the end effector 102 within the support 204, the support 204 may include one or more end effector retaining tabs 224 configured to selectively engage with an adjacent portion of the end effector 102 when the end effector 204 is disposed in the support 204. The one or more retaining tabs 224 may be configured to be cammed into engagement with the end effector 102 as the end effector 102 is inserted through the opening 228 of the support 204. Once engaged with the end effector, the one or more retaining tabs 224 may then apply a first retaining force to the end effector 102 that resists removal of the end effector 102 from the support 204 as well as rotation of the end effector 120 within the support 204 between the rails 230. In the depicted embodiment, the one or more retaining tabs 224 are connected to and extend from the segment 238 extending between the opposing rails 230. For example, the one or more retaining tabs may be a pair of retaining tabs located on opposing connecting segments on either side of the end effector 102 when the end effector 102 is disposed in the support. The one or more retaining tabs 224 may extend from the corresponding connecting segments 238 towards the base 238 and may either be angled inwards towards the end effector and/or may include a catch, hook, or other structure configured to be engaged with the end effector 102 to apply the above noted first retaining force.

As noted previously, it may be desirable to help avoid unintentional movement of a macroencapsulation device 400 within the internal cavity 116 of an end effector 102. While an end effector 102 may include a macroencapsulation device retaining tab 142 (see FIG. 7B) to aid in unintentional displacement of the macroencapsulation device out of the distal outlet, it may be desirable for a support 204 to also be configured to help mitigate or prevent unintentional movement of a macroencapsulation device 400 within an end effector 102 disposed within the support 204. In one such embodiment, one or more projections 208 may extend from an end portion of the rails 230 and/or a segment 238 extending between the rails 230. The one or more projections 208 may be configured such that they extend into a distal outlet 138 of the end effector 102 when the end effector 102 is disposed in the support 204. These one or more projections 208 may either extend for a portion, or an entire, external perimeter of the distal outlet 138 of the end effector 102. In either case, the one or more projections 208 may prevent movement of a macroencapsulation device 400 distally out of the distal outlet 138 of the end effector 102 while it is disposed in the support 204. Thus, the support 204 may be used to maintain both a pose of the end effector 102 and macroencapsulation device 400 within an internal volume of a container 202.

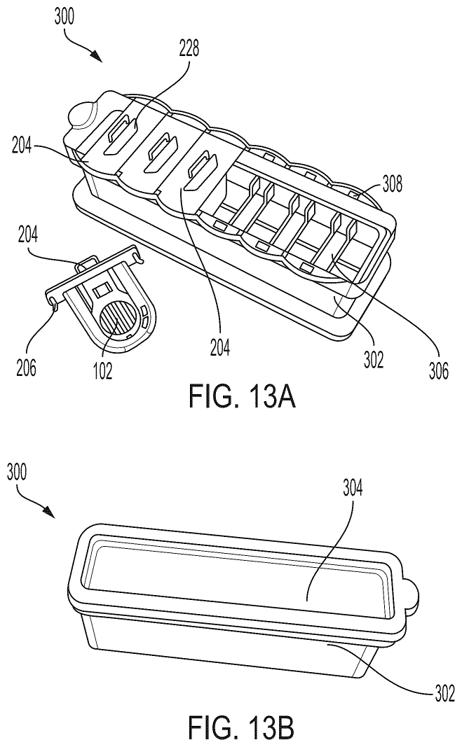

During an implantation procedure, it may be desirable to expose a macroencapsulation device to one or more media. For example, one or more washes may be applied to a macroencapsulation device prior to implantation to remove waste, other media the macroencapsulation device has been exposed to, and/or any other materials. FIG. 13A-13B depict a wash system 300. The wash system 300 may include a wash container 302 configured to contain a desired liquid such as one or more wash media. A wash container lid 304 may be appropriately sized and shaped to cover an opening of the wash container 302 when assembled thereon. The wash container 302 may include one or more receptacles 306 where each receptacle is configured to receive a corresponding support 204 disposed therein. When disposed in a corresponding receptacle, a support 204 may be appropriately positioned to expose an end effector and the macroencapsulation device disposed therein to the liquid contained in the wash container 302.

After being exposed to one or more desired treatments a user may remove an end effector 102 from the wash container 302 by attaching an elongated shaft of an implantation system, as described earlier, inserted through an opening 228 of the corresponding support 204. However, depending on the retention forces applied to the end effector contained within a support 204, when a user applies a force to remove the end effector from the support 204, the support 204 may be removed from the wash container 302 with the end effector. Correspondingly, as previously described, the one or more supports 204 may include one or more retention tabs 206, or other appropriate connectors, configured to connect a corresponding support 204 to the wash container 302. For example, correspondingly slots 308 may be formed on opposing sides of the wash container 302 such that a pair of opposing retention tabs 206 of a support 204 may be inserted into the slots 308 when the support 204 is inserted into a receptacle 308. To provide the desired retention of the support 204 in the wash container while permitting removal of the end effector from the support 204, the retaining force from the one or more retention tabs 206 holding the support 204 in the receptacle 306 may be greater than the retaining force from the end effector retaining tab 224 holding an end effector 102 in the support 204. With these appropriately balanced forces, an end effector 102 may be removed from the support 204 by an implantation system while the support 204 may be maintained stationary relative to the container 302. After removal from the wash container 302 and support 204, the end effector and macroencapsulation device may be used in an implantation procedure.

While a pair of corresponding retention tabs 206 and slots 308 have been depicted in the figure to provide the desired connection between a support 204 and receptacle of a wash container 302, it should be understood that any appropriate type of connection exhibiting the desired combination of forces may be used. Appropriate types of connections may include, but are not limited to, detents, magnetic connections, latches, threaded connections, interlocking mechanical features, and/or any appropriate type of connection as the disclosure is not limited in this fashion.

As stated above, and as illustrated in the figures, the wash system may be configured to house multiple supports 204 and end effectors 102. However, embodiments in which a single receptacle 306 is present in a wash container 302 for use with a single support 204 are also contemplated.

FIGS. 14A-14C show an embodiment of a macroencapsulation device 400 which may be used with any one of the embodiments of a macroencapsulation implantation system and/or container system disclosed herein. As shown in the figures, a macroencapsulation device 400 may comprise a first membrane layer 422 and a second membrane layer 418. In some embodiments, the first and/or second membrane layer may comprise a polymer material, such as expanded polytetrafluorethylene (ePTFE). In various embodiments, each of the first membrane layer 422 and the second membrane layer 418 may be either sintered or unsintered. Each of the first and second membrane layers may also comprise a single layer or multiple layers.

The first and second membrane layers 418 and 422 may be bonded together at a bonded perimeter 408 and optional bonded portions 410 located within the bonded perimeter. In FIG. 14A, a top surface of the second membrane layer 418 is shown with a bonded perimeter 408 of the membranes (e.g., where first and the second membrane layers are bonded) extending around a perimeter of the membranes. The bonded perimeter 408 may form an interior volume disposed between the first and second membrane layers configured to encapsulate a population of cells. In some embodiments, the bonded perimeter 408 may extend entirely around the perimeter of the membrane; however, as shown in FIG. 14A, the bonded perimeter may have an unbonded portion 402, for example to accommodate and/or cooperate with a fill port through which a population of cells may be introduced into an interior volume of the device. As will be appreciated, the dimensions of the bonded perimeter 408 may at least partially define a size of the interior volume. For example, in embodiments in which the membrane(s) and/or device have a generally circular shape, the bonded perimeter 408 may have a diameter 416, which may at least partially define a size of an interior volume between the first and second membrane layers.

In some embodiments, an interior volume between the first and second membrane layers may include a network of continuous interconnected volumes formed by and/or between the various bonded portions of the membrane. For example, as shown in FIG. 14C, an interior volume between the first membrane layer 422 and the second membrane layer 418 may include a network of interconnected volumes (e.g., channels 414) formed between the bonded portions 410. In some embodiments, an interior volume may have a volume thickness 426, which may be a maximum distance between the first and second membrane layers in a direction perpendicular to a maximum transverse dimension of the device. As will be appreciated, a total thickness of the device (e.g., the total thickness 430), may depend at least in part on the internal volume thickness 426, as well as the membrane layer thickness 428 for each membrane layer. As described herein, a thickness may be measured in a direction perpendicular to a maximum transverse dimension of the device.

As shown in the figures, the bonded perimeter may be disposed radially inward from the outer perimeter 420 of the membranes. The bonded portions 410 may take the form of bonded dots distributed across a surface area of the membranes in a hexagonal array. However, any appropriate shape, arrangement, configuration, and/or spacing of these bonded regions may also be used. For example, as shown in FIG. 14C, a bond spacing 432 may be a distance between adjacent bonded portions 410. Additionally, in some embodiments, one or more bonded portions 410 may include through holes 412 formed therein. Each through hole may have a through hole diameter 424. Due to the presence of these bonded regions located radially inwards from a bonded perimeter of the membranes, an internal volume formed between the membranes, once in the filled configuration (e.g., as shown in FIG. 14C), may take the form of a plurality of interconnected channels 414 corresponding to the unbonded regions of the membranes extending between these bonded portions.

In some embodiments, when the membrane layers are connected to a frame 406 that extends at least partially and, in some embodiments entirely around, the bonded perimeter 408 of the membranes. The unbonded portion 402 of the membranes may be positioned and sealed around a fill port 404 of the frame such that the fill port remains in fluid communication with the interior volume. In some embodiments, a fill port 404 may be included in the frame 406 to allow fluid communication in at least one direction between an external environment and the interior volume of the device. For example, a fill port 404 may be configured to allow the population of cells to be introduced into the volume between the first and second membrane layers 422 and 418. The fill port 404 may include a through hole, not depicted, extending through the fill port to an interior volume of the macroencapsulation device 400 formed by the first and second membrane layers 422 and 418 shown in FIGS. 14A-14C above.

In various embodiments, a frame 406 may be formed in any appropriate shape, including any appropriate round, elongated, rectilinear, polygonal (e.g., pentagonal, hexagonal, octagonal, etc.), and/or any other appropriate regular or irregular shape. For example, in the embodiment shown, the frame 406 may be formed in a generally circular shape. A frame thickness may be a maximum thickness between any two opposing surfaces or points of a cross-section of the frame. In some embodiments, a frame thickness may be measured in a direction perpendicular to a maximum transverse dimension of the frame 406 and/or the device 400.

While the above embodiment of a macroencapsulation device may be used with any of the macroencapsulation implantation systems and/or containers, it should be understood that the various embodiments disclosed herein are not limited to being used with such a device. Instead, the various embodiments of macroencapsulation implantation system and/or containers disclosed herein may be used with any appropriate type of macroencapsulation device as the disclosure is not limited in this fashion.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1.-67. (canceled)

68. An end effector for implanting a macroencapsulation device, the end effector comprising:
a neck configured to be attached to an elongated shaft of an implantation system;
a body attached to and extending out from a distal end portion of the neck, wherein a maximum transverse dimension of the neck in a direction perpendicular to a longitudinal axis of the end effector is less than a maximum transverse dimension of the body in the direction perpendicular to a longitudinal axis of the end effector;
a cavity formed in an interior of the body, wherein the cavity is sized and shaped to receive the macroencapsulation device therein;
a first opening formed on a proximal end portion of the neck;
a channel extending from the first opening to the cavity, wherein the first opening and the channel are configured to receive a pusher of the implantation system inserted therethrough; and
an outlet formed on a distal portion of the body, wherein the outlet is sized and shaped to permit the macroencapsulation device to be displaced out of the cavity through the outlet.

69. The end effector of claim 68, wherein a maximum transverse dimension of the cavity is greater than a maximum transverse dimension of the channel.

70. The end effector of claim 68, further comprising a plurality of slots formed on one or more surfaces of the body, wherein a long axis of each slot is aligned with the longitudinal axis.

71. The end effector of claim 70, wherein at least a portion of the plurality of slots include one or more fins extending from an exterior portion of the body towards the cavity.

72. The end effector of claim 68, further comprising a first portion of a connection formed on the neck.

73. The end effector of claim 72, wherein the first portion of the connection comprises a second opening configured to be connected to a catch of the implantation system.

74. The end effector of claim 68, wherein a maximum transverse dimension of the outlet is greater than or equal to a maximum transverse dimension of the cavity.

75. The end effector of claim 68, further comprising a pair of opposing ledges formed on opposing sides of the outlet.

76. The end effector of claim 68, further comprising one or more retaining tabs configured to apply a retaining force to a frame of the macroencapsulation device when the macroencapsulation device is disposed in the cavity of the end effector.

77. The end effector of claim 76, wherein the one or more retaining tabs are oriented in a distal direction and extend from the neck into the cavity.

78. The end effector of claim 68, wherein a transverse cross section of the body perpendicular to the longitudinal axis includes a flat portion and a non-linear portion.

79. The end effector of claim 78, wherein the non-linear portion is curved.

80. The end effector of claim 68, wherein at least a portion of a cross section of the cavity in a plane parallel to the longitudinal axis of the end effector is round.

81. The end effector of claim 68, further comprising the macroencapsulation device disposed in the cavity.

82. The end effector of claim 81, further comprising a population of cells disposed in the macroencapsulation device.

83. The end effector of claim 82, wherein the cell population is an insulin secreting population of cells.

84. A macroencapsulation device implantation system comprising:
an elongated shaft including an internal channel extending at least partially through and along a longitudinal axis of the elongated shaft;
a handle including a pusher slidably disposed within the internal channel of the elongated shaft; and
a first portion of a connection formed on a distal end portion of the elongated shaft, wherein the distal end portion of the elongated shaft and the pusher are configured to be inserted into an opening of an end effector to connect the end effector to the distal end portion of the elongated shaft, and wherein the elongated shaft is configured to be displaced proximally relative to the pusher to displace a macroencapsulation device out of an outlet of a cavity of the end effector with the pusher.

85. The system of claim 84, wherein the first portion of the connection includes a cam configured to cam inwards during insertion in the opening of the end effector.

86. The system of claim 84, wherein a distal portion of the pusher is configured to compliment a size and shape of a frame of the macroencapsulation device.

87. The system of claim 84, further comprising an actuation lock configured to prevent relative movement of the pusher and the elongated shaft in a locked configuration and permit relative movement of the pusher and the elongated shaft in an unlocked configuration.

88. The system of claim 87, wherein the actuation lock includes a depressible button, wherein depressing the depressible button moves the actuation lock to the unlocked configuration, and wherein displacing the depressible button proximally relative to the handle moves the elongated shaft and the end effector proximally relative to the pusher when the end effector is connected to the elongated shaft.

89. The system of claim 88, wherein the handle comprises a slot sized and shaped to accommodate proximal movement of the depressible button.

90. The system of claim 89, further comprising a protrusion formed on the elongated shaft, wherein the protrusion is disposed outside of the slot when the elongated shaft is in an extended configuration and wherein the protrusion is disposed inside of the slot when the elongated shaft is in a retracted configuration.

91. The system of claim 88, wherein the actuation lock includes one or more protrusions operatively coupled with a trigger, and wherein a blocking portion of the pusher is configured to prevent movement of the one or more protrusions when the trigger is in a locked configuration and permit movement of the one or more protrusions in a direction parallel to the longitudinal axis in the unlocked configuration.

92. The system of claim 91, wherein actuating the trigger moves the one or more protrusions out of alignment with the blocking portion to move the actuation lock from the locked configuration to the unlocked configuration.

93. The system of claim 84, further comprising the end effector.

94. The system of claim 93, further comprising a second portion of the connection disposed on the end effector, wherein the second portion of the connection is configured to be connected to the first portion of the connection.

95. The system of claim 93, further comprising the macroencapsulation device disposed in the end effector.

96. The system of claim 95, further comprising a population of cells disposed in the macroencapsulation device.

97. The system of claim 96, wherein the population of cells is an insulin secreting population of cells.

98. A method of implanting a macroencapsulation device, the method comprising:
inserting a distal end portion of an elongated shaft including a first portion of a connection and a pusher into an opening of an end effector including the macroencapsulation device disposed therein;
engaging the first portion of the connection with a second portion of the connection formed on the end effector to connect the end effector to the elongated shaft;
contacting the pusher with the macroencapsulation device; and
proximally displacing the elongated shaft and the end effector relative to the pusher and the macroencapsulation device to displace the macroencapsulation device out of a distal outlet of the end effector.

99.-111. (canceled)

112. A macroencapsulation device implantation system comprising:
an end effector comprising:
a neck;
a body attached to and extending out from a distal end portion of the neck, wherein a maximum transverse dimension of the neck in a direction perpendicular to a longitudinal axis of the implantation system is less than a maximum transverse dimension of the body in the direction perpendicular to the longitudinal axis of the implantation system;
a cavity formed in an interior of the body, wherein the cavity is sized and shaped to receive a macroencapsulation device therein; and
an outlet formed on a distal portion of the body, wherein the outlet is sized and shaped to permit the macroencapsulation device to be displaced out of the cavity through the outlet;
an elongated shaft, wherein a distal end portion of the elongated shaft is attached to the end effector, and wherein a maximum transverse dimension of the elongated shaft in the direction perpendicular to the longitudinal axis of the implantation system is less than the maximum transverse dimension of the body;
an internal channel extending at least partially through and along a longitudinal axis of the elongated shaft to the cavity; and
a handle including a pusher slidably disposed within the internal channel of the elongated shaft, wherein the end effector and the elongated shaft are proximally displaceable relative to the pusher.

113.-128. (canceled)
